(19) United States
(12) Patent Application Publication
(10) Pub. No.: US 2020/0078418 A1
(43) Pub. Date: Mar. 12, 2020

(54) TARGETED THERAPEUTIC COMPOSITIONS AND METHODS OF USE THEREOF FOR CLOSTRIDIUM DIFFICILE INFECTIONS

(71) Applicant: Arizona Board of Regents on Behalf of Northern Arizona University, Flagstaff, AZ (US)

(72) Inventors: David M. Wagner, Flagstaff, AZ (US); Nathan E. Stone, Flagstaff, AZ (US); Emily K. Cope, Flagstaff, AZ (US); Jason W. Sahl, Flagstaff, AZ (US); Fernando P. Monroy, Flagstaff, AZ (US); Paul S. Keim, Flagstaff, AZ (US); Joseph D. Busch, Flagstaff, AZ (US)

(21) Appl. No.: 16/562,378

(22) Filed: Sep. 5, 2019

Related U.S. Application Data
(60) Provisional application No. 62/729,331, filed on Sep. 10, 2018.

(51) Int. Cl. A61K 35/741; A61K 45/06

(52) U.S. Cl. CPC A61K 35/741; A61K 45/06

(57) ABSTRACT

Compositions and methods of use thereof for the treatment of disorders associated with the gastrointestinal tract, including Clostridium difficile infections are provided for use in, e.g., the fields of medicine and gastroenterology.

### INCORPORATION BY REFERENCE TO RELATED APPLICATION

Any and all priority claims identified in the Application Data Sheet, or any correction thereto, are hereby incorporated by reference under 37 CFR 1.57. This application claims the benefit of U.S. Provisional Application No. 62/729,331, filed on Sep. 10, 2018. The aforementioned application is incorporated by reference herein in its entirety, and is hereby expressly made a part of this specification.

### FIELD

Compositions and methods of use thereof for the treatment of disorders associated with the gastrointestinal tract, including Clostridium difficile infections are provided for use in, e.g., the fields of medicine and gastroenterology.

### BACKGROUND

Marked by an increase in disease severity and recurrence since the early 2000s, Clostridioides (Clostridium) difficile infection (CDI) has rapidly become an emerging public health threat. Clostridioides difficile is the most common cause of antimicrobial associated diarrhea in healthcare facilities worldwide and is the leading cause of hospital-associated infections in the United States, resulting in an estimated 14,000 deaths every year. Clostridioides difficile is a Gram positive, spore-forming, obligate anaerobe that can colonize, proliferate, and produce devastating toxins in the human gastrointestinal tract. Not all forms of C. difficile are capable of causing disease; certain strains are non-toxigenic due to the absence of the 19.6 kb pathogenicity locus (PaLoc), which contains one or two toxin-encoding (tcdA and tcdB) and three regulatory (tcdA, tcdR, tcdE) genes. Toxigenic strains excrete two major exotoxins (TcdA and TcdB; hereafter toxins), are genetically and phenotypically highly diverse, and can cause a range of symptoms, including: mild intestinal discomfort, pseudomembranous colitis, toxic megacolon, colonic perforation (leading to organ failure), and death.

Hospital acquisition of C. difficile is well documented, but the increased role and importance of community-acquired infections has only recently been described and has yet to be fully understood. Several community and environmental reservoirs for this pathogen have been suggested, including farm animals, food products, companion pets, soil, and water; genetic analyses of paired C. difficile isolates collected from humans and some of these community sources have suggested that transmission between them was plausible. Previous work revealed that >10% of sampled domestic canines in Flagstaff, Ariz. carried strains of C. difficile that were genetically similar to those known to cause human disease. Because 36.5% of households in the US are estimated to own domestic canines, and an appreciable proportion of canines have been shown to carry C. difficile, the possibility of transmission between humans and canines is quite plausible.

In humans and canines, deviations from microbial homeostasis of the gut are known to be associated with gastrointestinal disease and can be observed by comparing the microbial gut composition of healthy and diseased individuals using 16S rRNA gene sequencing. Studies of the human gut microbiome have revealed moderate to severe dysbiosis (i.e. compositional shifts in the bacterial community) associated with C. difficile disease and although studies that directly explore this association in canines have yet to be conducted, a recent review describes the microbial composition of the healthy canine gut and several studies have shown that dysbiosis in canines is observed in association with inflammatory bowel disease and acute diarrhea. Broadly speaking, deviations from homeostasis are observed in diseased individuals and, upon recovery, microbiome diversity is generally restored. Interestingly, asymptomatic colonization of C. difficile in humans has also been documented, with carriage rates estimated at 60-70% in infants and 3% in healthy adults. This rate can be even more pronounced in clinical settings where as many as 51% of healthy individuals have been reported to be colonized with toxigenic C. difficile.

Treatment options for severe and recurrent human CDI aimed at restoring a healthy gut microbiome have recently included microbial replacement therapy via fecal microbiome transplants (FMT). FMT therapies have yielded extremely positive outcomes, boasting success rates>90% and, thus, have reinforced the role of the healthy gut microbiome in CDI resistance. However, the exact mechanisms and microbial interactions underlying these successes have yet to be fully understood. Furthermore, this therapy is invasive, not universally approved as a treatment option, and carries with it the risk of unintentional bacterial or viral infections stemming from incomplete screening of the donor sample. As such, targeted therapeutics aimed at restoring the function of a healthy gut microbiome that are approvable for all patients with CDI are needed

Together, the rate of asymptomatic C. difficile carriage in humans, the ubiquitous connection of CDI onset and antibiotic use, and the impressive rate of recovery associated with microbiome replacement therapies suggests that: 1) protective microbes are present in the healthy gut, 2) these protective microbes are reduced or eliminated as a result of antibiotic usage, and 3) it should be possible to identify these microbes and the underlying mechanisms, which would facilitate the development of targeted therapeutics aimed at restoring C. difficile colonization and infection resistance.

Accordingly, a system and method are provided for a targeted therapy to C. difficile infection and potential for infection resistance is provided.

In a generally applicable first aspect (i.e., independently combinable with any of the aspects or embodiments identified herein), a composition is provided comprising at least one bacterium selected from the genera consisting of Clostridium and/or Sphingobacterium.

In a generally applicable embodiment of the first aspect (i.e. independently combinable with any of the aspects or embodiments identified herein), the composition comprises at least one bacterium selected from the genera Clostridium and at least one bacterium selected from the genera Sphingobacterium.

In a generally applicable embodiment of the first aspect (i.e. independently combinable with any of the aspects or embodiments identified herein), the composition comprises Clostridium hiranon.

In a generally applicable embodiment of the first aspect (i.e. independently combinable with any of the aspects or embodiments identified herein), the composition comprises Sphingobacterium faecium.

In a generally applicable embodiment of the first aspect (i.e. independently combinable with any of the aspects or embodiments identified herein), the composition comprises Clostridium hiranon and Sphingobacterium faecium.

In a generally applicable embodiment of the first aspect (i.e. independently combinable with any of the aspects or embodiments identified herein), the composition is formulated for oral or rectal administration to a mammalian subject.

In a generally applicable embodiment of the first aspect (i.e. independently combinable with any of the aspects or embodiments identified herein), the composition is selected from the group consisting of: a probiotic composition or product combination, a pharmaceutical composition or product combination, a dietary supplement, and a food; or a combination of two or more of the listed items.

In a generally applicable embodiment of the first aspect (i.e. independently combinable with any of the aspects or embodiments identified herein), the composition comprises at least two bacteria, wherein each of the bacteria is provided in a different composition.

In a generally applicable embodiment of the first aspect (i.e. independently combinable with any of the aspects or embodiments identified herein), the composition comprises 10⁴ cfu or more of the at least one bacterium.

In a generally applicable embodiment of the first aspect (i.e. independently combinable with any of the aspects or embodiments identified herein), the composition comprises less than 10⁴ cfu of the at least one bacterium.

In a generally applicable embodiment of the first aspect (i.e. independently combinable with any of the aspects or embodiments identified herein), the composition comprises from 10⁴ cfu to 10⁶ cfu of the at least one bacterium.

In a generally applicable embodiment of the first aspect (i.e. independently combinable with any of the aspects or embodiments identified herein), the composition comprises 10⁶ cfu or more of the at least one bacterium.

In a generally applicable embodiment of the first aspect (i.e. independently combinable with any of the aspects or embodiments identified herein), the composition is for use in treating, preventing, ameliorating, or decreasing a likelihood of a Clostridium difficile infection in a subject.

In a generally applicable second aspect (i.e. independently combinable with any of the aspects or embodiments identified herein), a method is provided of treating, preventing, or ameliorating a Clostridium difficile infection in a subject in need thereof, the method comprising: administering to the subject the composition of the first aspect or any of its embodiments to a subject in need thereof, whereby a Clostridium difficile infection is treated, prevented, or ameliorated.

In a generally applicable embodiment of the second aspect (i.e. independently combinable with any of the aspects or embodiments identified herein), the Clostridium difficile infection is treated.

In a generally applicable embodiment of the second aspect (i.e. independently combinable with any of the aspects or embodiments identified herein), the Clostridium difficile infection is prevented.

In a generally applicable embodiment of the second aspect (i.e. independently combinable with any of the aspects or embodiments identified herein), the Clostridium difficile infection is ameliorated.

In a generally applicable embodiment of the second aspect (i.e. independently combinable with any of the aspects or embodiments identified herein), the subject is infected with Clostridium difficile.

In a generally applicable embodiment of the second aspect (i.e. independently combinable with any of the aspects or embodiments identified herein), the subject is not infected with Clostridium difficile but is exposed to Clostridium difficile before the administering or after the administering.

In a generally applicable embodiment of the second aspect (i.e. independently combinable with any of the aspects or embodiments identified herein), the method further comprises prior to the administering, selecting the subject as being within a class of subjects that are in need of receiving the composition.

In a generally applicable embodiment of the second aspect (i.e. independently combinable with any of the aspects or embodiments identified herein), the selecting comprises identifying the subject as having a Clostridium difficile infection.

In a generally applicable embodiment of the second aspect (i.e. independently combinable with any of the aspects or embodiments identified herein), the method further comprises administering an antibiotic to the subject prior to administering the composition or product combination, said antibiotic reducing a total quantity of gut bacteria of the subject by at least 80% prior to the administering of the composition.

In a generally applicable embodiment of the second aspect (i.e. independently combinable with any of the aspects or embodiments identified herein), the composition is in a form suitable for oral administration.

In a generally applicable embodiment of the second aspect (i.e. independently combinable with any of the aspects or embodiments identified herein), the composition is prepared as a probiotic for oral administration suitable as a dietary supplement.

In a generally applicable embodiment of the second aspect (i.e. independently combinable with any of the aspects or embodiments identified herein), the subject is human.

In a generally applicable embodiment of the second aspect (i.e. independently combinable with any of the aspects or embodiments identified herein), the subject is canine.

In a generally applicable third aspect (i.e., independently combinable with any of the aspects or embodiments identified herein), a composition is provided that is selected from the group consisting of a probiotic composition, a probiotic product combination, a pharmaceutical composition, a pharmaceutical product combination, a dietary supplement, a food, and combinations thereof, the composition comprising at least one bacterium selected from the genera consisting of Clostridium and/or Sphingobacterium, formulated for oral or rectal administration to a mammalian subject.

In a generally applicable embodiment of the third aspect (i.e. independently combinable with any of the aspects or embodiments identified herein), the composition comprises at least one bacterium selected from the genera Clostridium and at least one bacterium selected from the genera Sphingobacterium.

In a generally applicable embodiment of the third aspect (i.e. independently combinable with any of the aspects or embodiments identified herein), the composition comprises Clostridium hiranon.

In a generally applicable embodiment of the third aspect (i.e. independently combinable with any of the aspects or embodiments identified herein), the composition comprises Sphingobacterium faecium.

In a generally applicable embodiment of the third aspect (i.e. independently combinable with any of the aspects or embodiments identified herein), the composition comprises Clostridium hiranon and Sphingobacterium faecium.

In a generally applicable embodiment of the third aspect (i.e. independently combinable with any of the aspects or embodiments identified herein), the composition comprises at least at least two bacteria selected from the genera consisting of Clostridium and/or Sphingobacterium, wherein each of the bacteria is provided in a different composition.

In a generally applicable embodiment of the third aspect (i.e. independently combinable with any of the aspects or embodiments identified herein), the composition comprises 10⁴ cfu or more of the at least one bacterium.

In a generally applicable embodiment of the third aspect (i.e. independently combinable with any of the aspects or embodiments identified herein), the composition comprises 10⁶ cfu or more of the at least one bacterium.

In a generally applicable embodiment of the third aspect (i.e. independently combinable with any of the aspects or embodiments identified herein), the composition is in a form suitable for oral administration.

In a generally applicable embodiment of the third aspect (i.e. independently combinable with any of the aspects or embodiments identified herein), the composition is in a form for use as a probiotic for oral administration as a dietary supplement.

In a generally applicable embodiment of the third aspect (i.e. independently combinable with any of the aspects or embodiments identified herein), wherein the subject is human.

In a generally applicable embodiment of the third aspect (i.e. independently combinable with any of the aspects or embodiments identified herein), the subject is canine.

In a generally applicable fourth aspect (i.e., independently combinable with any of the aspects or embodiments identified herein), a method of treating, preventing, or ameliorating a Clostridium difficile infection is provided, comprising administering the composition of claim 1 to a subject in need thereof, whereby a Clostridium difficile infection is treated, prevented, or ameliorated.

In a generally applicable embodiment of the fourth aspect (i.e. independently combinable with any of the aspects or embodiments identified herein), the Clostridium difficile infection is treated.

In a generally applicable embodiment of the fourth aspect (i.e. independently combinable with any of the aspects or embodiments identified herein), the subject is infected with Clostridium difficile.

In a generally applicable embodiment of the fourth aspect (i.e. independently combinable with any of the aspects or embodiments identified herein), the subject is not infected with Clostridium difficile but is exposed to Clostridium difficile before administering the composition or after administering the composition.

In a generally applicable embodiment of the fourth aspect (i.e. independently combinable with any of the aspects or embodiments identified herein), the method further comprises prior to the administering the composition, selecting the subject as being within a class of subjects that are in need of receiving the composition.

In a generally applicable embodiment of the fourth aspect (i.e. independently combinable with any of the aspects or embodiments identified herein), the selecting comprises identifying the subject as having a Clostridium difficile infection.

In a generally applicable embodiment of the fourth aspect (i.e. independently combinable with any of the aspects or embodiments identified herein), the method further comprises administering an antibiotic to the subject prior to administering the composition, whereby the antibiotic reduces a total quantity of gut bacteria of the subject by at least 80% prior to the administering the composition.

### BRIEF DESCRIPTION OF THE DRAWINGS

The drawings described herein constitute part of this specification and includes exemplary embodiments of the present invention which may be embodied in various forms. It is to be understood that in some instances, various aspects of the invention may be shown exaggerated or enlarged to facilitate an understanding of the invention. Therefore, drawings may not be to scale.

FIGS. 1A-B depict transepithelial electrical resistance (TEER) results for human and canine colonic epithelial cells. TEER measurements are expressed as the proportion of the resistance observed at 0 hours; proportions were normalized to the untreated control cells (i.e., maximum TEER) for every time-point. FIG. 1A shows Caco-2 human epithelial cells are susceptible to C. difficile toxins derived from both canine and human isolates (p=0.009, Kruskal-Wallis). FIG. 1B shows Canine epithelial cells are susceptible to these same toxins (p=0.016, Kruskal-Wallis).

FIGS. 2A-C depict PCoA plots illustrating the differences between canine and human gut microbiomes, with and without the presence of Clostridioides difficile, based on Unweighted UniFrac distances. FIG. 2A shows the dysbiosis that distinguishes CDI humans from healthy humans is illustrated in this PCoA plot. FIG. 2B show canine samples do not cluster according to C. difficile status and do not display patterns of dysbiosis associated with C. difficile carriage as is observed in humans with CDI. FIG. 2C shows canine gut microbiomes are highly diverse and more similar to CDI humans than to healthy humans. P-values of <0.05 were considered significant.

FIGS. 3A-C depicts plot displaying the differences in alpha diversity between human and canine cohorts. FIG. 3A shows taxonomic differences between the four cohorts at the phylum level. FIG. 3B shows species richness and FIG. 3C shows Faith's phylogenetic diversity are decreased in the presence of C. difficile in humans, but less so in canines. P-values of <0.05 were considered significant.

FIGS. 4A-B depict three taxonomic groups that were differentially abundant in canines versus humans. FIG. 4A—Linear discriminant analysis (LDA) effect size (LefSe) analysis identified three bacterial taxa that were the major contributors to the differences observed between human and canine microbiome samples (bold text). FIG. 4B—The presence of Clostridium hiranonis and Sphingobacterium faecium was not significantly different (p-value>0.05) between the C. diff Canine (C. difficile positive) and Healthy Canine (C. difficile negative) cohorts, but the presence of Arthrobacter spp. was significantly more frequent in the Healthy Canine cohort. The C. diff Human cohort did not contain any reads from these three taxa; however, the Healthy Human cohort did contain C. hiranonis reads.

FIGS. 5A-D depict PCoA plots illustrating the differences associated with several canine fecal sample features, based on Unweighted UniFrac distances. Canine fecal samples did not cluster according to FIG. 5A Clostridioides difficile toxin status (p=0.479, PERMANOVA), FIG. 5B stool consistency (p=0.289, PERMANOVA), FIG. 5C sampling source (p=0.198, PERMANOVA), or FIG. 5D sampling method (p=0.072, PERMANOVA).

FIGS. 6A-C—maximum parsimony mid-point rooted phylogeny paired with SDS-PAGE and western blot data reveal differences in toxin production among seven C. difficile isolates from diverse sources. FIG. 6A—Maximum parsimony phylogeny created using whole genome SNPs discovered by aligning six sequenced genomes against C. difficile genome ATCC® BAA1870™ illustrates genomic differences among isolates. FIG. 6B—Total Clostridioides difficile extracellular protein content after 5 days of growth in BHIS media was separated using SDS-PAGE and detected by SYPRO® Ruby protein gel stain. Gel lanes were re-arranged post analysis in Adobe® Illustrator to align with the phylogeny presented in panel A. FIG. 6C—Western blot of extracellular C. difficile toxins (TcdA and TcdB) from seven C. difficile isolates after 5 days of growth in BHIS media. Gel lanes were re-arranged post analysis to align with the phylogeny presented in panel A.2.

FIGS. 7A-B depicts additional C. difficile derived toxins (combined TcdA and TcdB) that were tested using transepithelial electrical resistance (TEER). FIG. 7A Caco-2 human and FIG. 7B canine epithelial cells are susceptible to C. difficile toxins derived from canine, human, and environmental isolates, but tight junction breakdown differences between isolates did not correspond to multilocus sequence type (ST) (Caco-2 p=0.002, canine p=0.013, Kruskal-Wallis).

FIG. 8 depicts a C. difficile toxin dilution curve. Serially diluted C. difficile toxins (TcdA and TcdB) derived from the highly toxigenic control strain (ST-1: toxigenic control, ATCC® BAA1870™) demonstrated that reductions in TEER measurements (i.e., tight junction breakdown) were toxin dose dependent.

FIG. 9A-D depicts an analysis of Analysis of C. difficile negative vs. C. difficile positive healthy humans. FIG. 9A—PCoA plot of Unweighted UniFrac distances and FIG. 9B—taxonomic assignment at the phylum level revealed that Healthy Human cohort samples cluster together and have highly similar microbial compositions regardless of C. difficile status (Healthy Human C. diff e vs. Healthy Human C. diff+), but are highly dissimilar from CDI Human cohort samples. FIG. 9C—Species richness and FIG. 9D Faith's phylogenetic diversity are not significantly different between the Healthy Human cohort subgroups, but both subgroups are significantly different from the CDI Human cohort. P-values or q-values of <0.05 were considered significant.

FIGS. 10A-B depict analysis of a second C. difficile infection (CDI) human dataset. The inclusion of additional 16S rRNA gene sequences from CDI human fecal samples (n ¼ 88) demonstrated that the clustering and compositional patterns of fecal microbiomes presented in this study are the result of host and C. difficile status differences rather than biases associated with the use of sequences processed by multiple lab groups/facilities. FIG. 10A—PCoA plot of Unweighted UniFrac distances and FIG. 10B—taxonomic barplots collapsed at the phylum level for five 16S rRNA gene datasets processed at three different facilities.

FIG. 10C depicts the most likely number of clusters represented in three datasets as identified by silhouette width scores using Unweighted UniFrac distances in the partitioning around medoids (PAM) clustering algorithm. Clustering solutions of 2, 3, and 4 were suggested as plausible and warranted further investigation.

FIGS. 11A-B depict clustering of samples as identified using PAM. FIG. 11A Clustering solutions of 2, 3, and 4 are represented to illustrate the differences between human and canine microbiomes as well as the associations of Clostridioides difficile status in both species. FIG. 11B Principle component analysis (PCA) plots of PAM clusters at k¼ 2, 3, and 4.8.

FIGS. 12A-D depict linear discriminant analysis (LDA) effect size (LefSe) identified differentially abundant taxa that distinguished cohorts. FIG. 12A—The major taxa that differentiate the combined canine cohorts (Canine C. diff+ and Canine C. diff− cohorts binned together) from the CDI Human cohort are reflected in this plot. FIG. 12B—The major taxa that differentiate the combined canine cohorts from the Healthy Human cohort are reflected in this plot. FIG. 12C The major taxa that differentiate the CDI Human cohort from the Healthy Human cohort are reflected in this plot. FIG. 12D The major taxa that differentiate the Canine C. diff+ cohort from the Canine C. diff− cohort are reflected in this plot.

### DETAILED DESCRIPTION OF EMBODIMENTS

The following description and examples illustrate embodiments of the present invention in detail. Those of skill in the art will recognize that there are numerous variations and modifications of this invention that are encompassed by its scope. Accordingly, the description of an embodiment should not be deemed to limit the scope of the present invention.

Clostridioides difficile infection (CDI) is an emerging public health threat and C. difficile is the most common cause of antimicrobial-associated diarrhea worldwide and the leading cause of hospital-associated infections in the US, yet the burden of community-acquired infections (CAI) is poorly understood. Characterizing C. difficile isolated from canines is important for understanding the role that canines may play in CAI. In addition, several studies have suggested that canines carry toxigenic C. difficile asymptomatically, which may imply that there are mechanisms responsible for resistance to CDI in canines that could be exploited to help combat human CDI. To assess the virulence potential of canine-derived C. difficile, we tested whether toxins TcdA and TcdB (hereafter toxins) derived from a canine isolate were capable of causing tight junction disruptions to colonic epithelial cells. Additionally, we addressed whether major differences exist between human and canine cells regarding C. difficile toxicity by exposing them to identical toxins. We then examined the canine gut microbiome associated with C. difficile carriage using 16S rRNA gene sequencing and searched for deviations from homeostasis as an indicator of CDI. Finally, we queried 16S rRNA gene sequences for bacterial taxa that may be associated with resistance to CDI in canines. Clostridioides difficile from a canine produced toxins that reduced tight junction integrity in both human and canine cells in vitro. However, canine guts were not dysbiotic in the presence of C. difficile. These findings support asymptomatic carriage in canines and, furthermore, suggest that there are features of the gut microbiome and/or a canine-specific immune response that may protect canines against CDI. Two biologically relevant bacteria that may aid in CDI resistance in canines have been identified: 1) Clostridium hiranonis, which synthesizes secondary bile acids that have been shown to provide resistance to CDI in mice; and 2) Sphingobacterium faecium, which produces sphingophospholipids that may be associated with regulating homeostasis in the canine gut. These findings suggest that canines may be cryptic reservoirs for C. difficile and, furthermore, that mechanisms of CDI resistance in the canine gut could provide insights into targeted therapeutics for human CDI.

Treatment options for severe and recurrent human CDI aimed at restoring a healthy gut microbiome have recently included microbial replacement therapy via fecal microbiome transplantation (FMT). FMT therapies have yielded extremely positive outcomes, boasting success rates>90% and, thus, have reinforced the role of the healthy gut microbiome in CDI resistance. However, the exact mechanisms and microbial interactions underlying these successes have yet to be fully understood. Furthermore, this therapy can be invasive, is not universally approved as a treatment option, and carries with it the risk of unintentional bacterial or viral infections stemming from incomplete screening of the donor sample. As such, targeted therapeutics aimed at restoring the function of a healthy gut microbiome that can be utilized by all patients with CDI are needed. Together, the rate of asymptomatic C. difficile carriage in humans, the ubiquitous connection of CDI onset and antibiotic use, and the impressive rate of recovery associated with microbiome replacement therapies suggests that: 1) protective microbes are present in the healthy gut, 2) these protective microbes are reduced or eliminated as a result of antibiotic usage, and 3) it should be possible to identify these microbes and the underlying mechanisms, which would facilitate the development of targeted therapeutics aimed at restoring C. difficile colonization and infection resistance.

Recent research has explored the mechanisms and interactions responsible for resistance to C. difficile colonization in the healthy human gut and characterized the underlying bacterial communities responsible for this phenotype. As a result, several mechanisms have been proposed, including: secondary bile acid mediation, mucosal carbohydrate competition, host immune defense activation, and antibacterial peptide production. One major challenge to this research has been the acquisition of samples from both non-colonized persons and asymptomatic C. difficile carriers that display the infection resistance phenotype, likely because healthy individuals are rarely sampled. To overcome this, we explored the use of canine fecal samples as a model for asymptomatic carriers and non-colonized hosts to make comparison with the human gut microbiome. There are three justifications for this approach: 1) canines carry C. difficile at an increased rate compared to humans (˜3% in healthy humans; ˜17% in healthy canines), so C. difficile positive samples will be more abundant; 2) although some canines may be susceptible to CDI, a majority appear to carry C. difficile asymptomatically (i.e. no diarrhea), suggesting a protective microbial community that may enlist a similar colonization or infection resistance mechanism to humans; and 3) samples are relatively easy to obtain and are not constrained by many of the regulatory hurdles common with human subject research.

Scientific support for the anecdotal observations of asymptomatic carriage in canines was established. The analysis of C. difficile in canines was approached in a step-wise fashion consisting of four major goals: 1) to experimentally confirm toxin production in canine-derived C. difficile and examine the potential of these toxins to cause human disease using an in vitro human colonic epithelial cell line model 2) to explore the possibility that canine colonic epithelial cells exhibit infection resistance when exposed to these same toxins by measuring the effects on canine and human epithelial cells, 3) to examine the canine gut microbiome associated with the presence of C. difficile using 16S rRNA gene sequencing to look for evidence of gut microbial differences (i.e. dysbiosis) associated with the presence of C. difficile in canines as an indicator of disease, and 4) to identify taxonomic differences between canine and human gut microbiomes that may be associated with asymptomatic carriage and/or colonization resistance in canines.

### Materials and Methods

C. difficile toxin challenge study design and sample selection was designed to validate that canine derived toxigenic C. difficile are virulent in cell culture and to facilitate comparisons to human isolates. A C. difficile strain collection (isolates from humans, canines, and the environment) and corresponding whole genome sequence (WGS) database was queried for genetically similar isolates derived from canines and humans. A clade was identified belonging to C. difficile sequence type (ST) 2 that contained a canine (ST-2: canine isolate, NCBI SRA # SRR3115498) and a human isolate (ST-2: human isolate, NCBI SRA # SRR6841703) that differed by only 53 single nucleotide polymorphisms (SNPs) out of 3,602,522 total nucleotide positions (0.0014%) that were used to build the phylogeny. In addition to these related isolates a high toxin producing ST-1 isolate (ST-1: toxigenic control, ATCC® BAA1870™, NCBI SRA # SRR7309420) and a nontoxigenic ST-3 canine isolate from our collection (ST-3: nontoxigenic control, NCBI SRA # SRR3115458) was included as toxin positive and negative controls, respectively. To further elucidate toxin production differences in closely related C. difficile strains from humans, canines, and the environment, also included were an environmental soil isolate (ST-2: soil isolate, NCBI SRA # SRR7309419) that differed from the ST-2: canine isolate by 27 SNPs (0.0007%), a second ST-2 human isolate (ST-2: human isolate 2, NCBI SRA # SRR7309421) that differed from the ST-2: canine isolate by 1291 SNPs (0.0358%), and a human ST-1 isolate (ST-1: human isolate, NCBI SRA # SRR6890723; difference from ST-1: toxigenic control=32 SNPs or 0.0008%). All sequenced genomes were aligned against finished C. difficile genomes CD630 (for ST-2 comparisons; Genbank accession # AM180355.1) and CD196 (for ST-1 comparisons; Genbank accession # FN538970.1) and SNPs were called in conjunction with NASP as described in our previous publication. All isolates were processed in identical fashion (regarding culturing conditions, toxin extractions, and epithelial cell treatments) to enable meaningful comparisons among them. Table 2 presents seven C. difficile isolates that were used to conduct the transepithelial electrical resistance (TEER) assays. Isolates representing three multilocus sequence types from clinical and environmental sources were used, including a high toxin producing control strain (ATCC® BAA1870™). Results are presented in three figures and Genbank SRA # s for each isolate are provided.

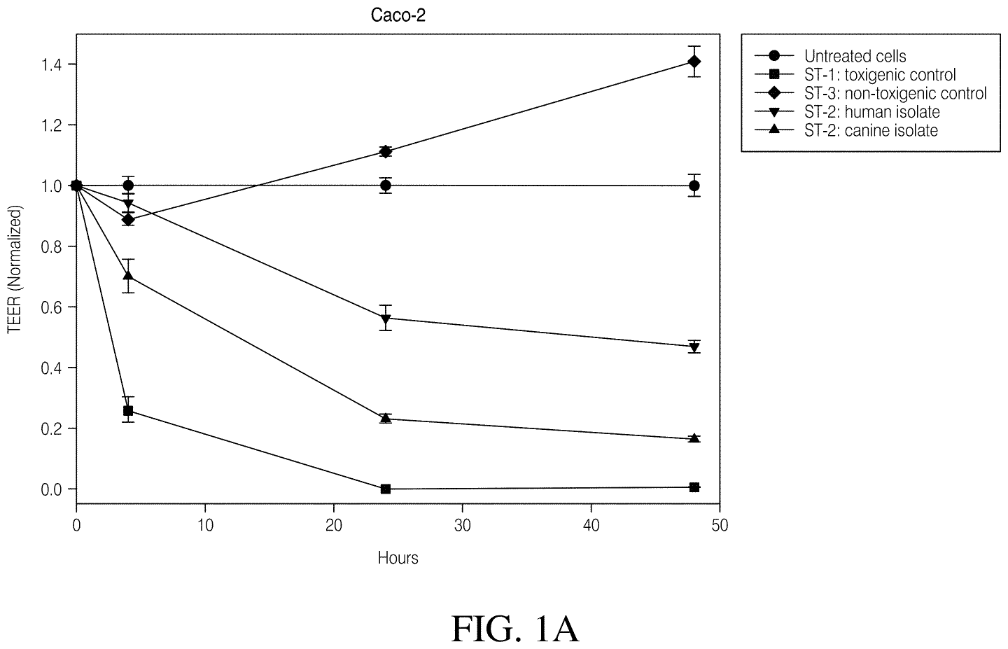

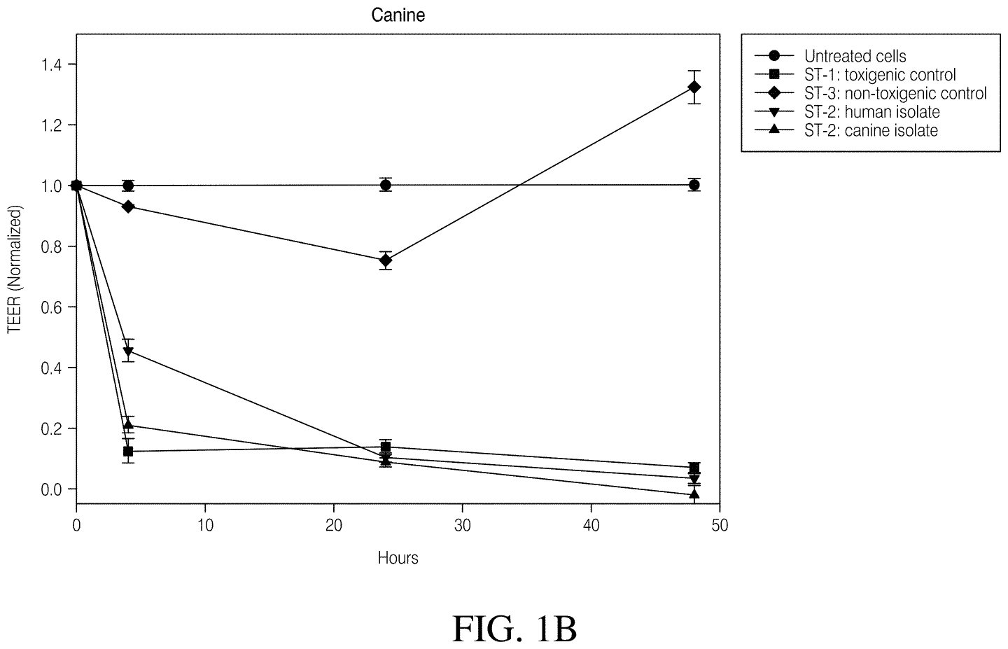

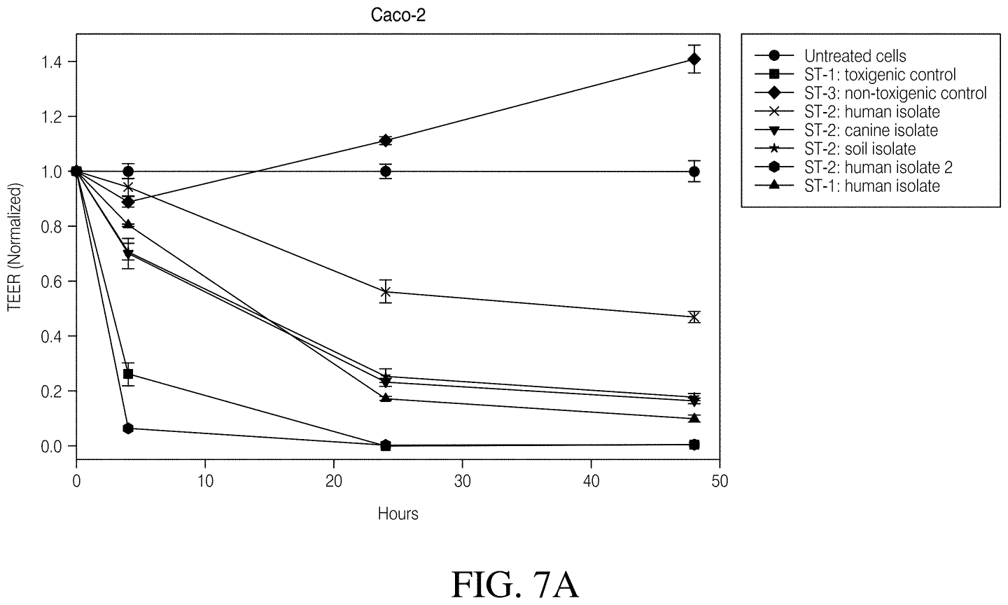

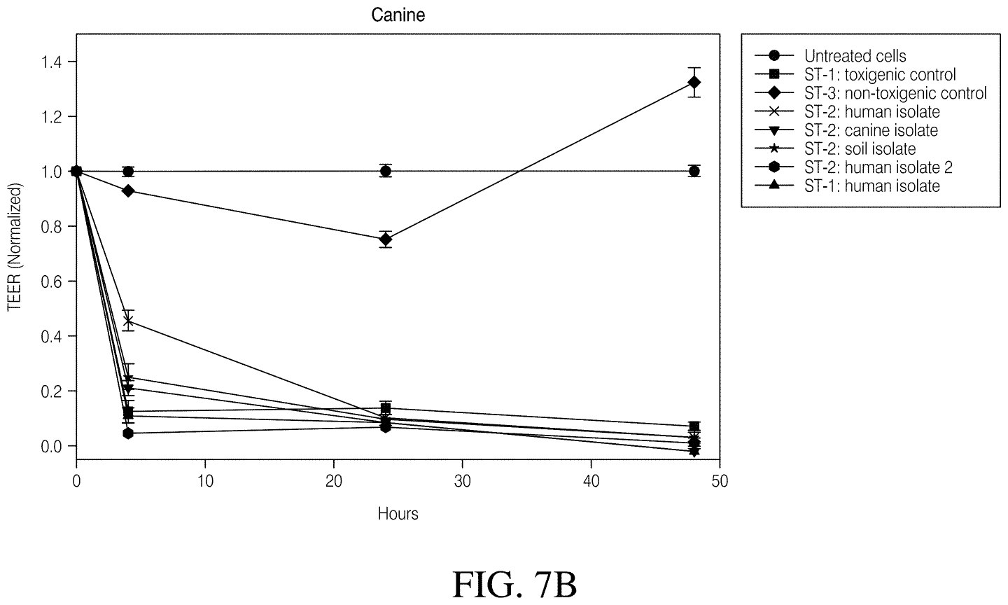

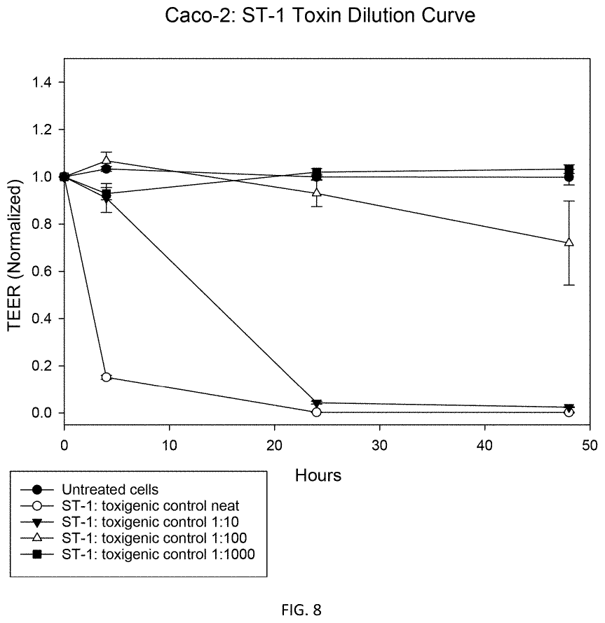

TABLE 2

| | | Multilocus | | | | |
|---|---|---|---|---|---|---|
| | | Sequence | Strain | | | Genbank |
| Isolate ID | Source | Type (ST) | designation | Reference | FIG. | SRA # |
| ST-1: | ATCC | ST-1 | ATCC ® | ATCC C. difficile | FIGS. 1A-B, | SRR7309420 |
| toxigenic | | | BAA1870 ™ | strain 4118 | FIGS. | |
| control | | | | | 7A-B, and | |
| | | | | | FIG. 8 | |
| ST-3: non- | Environmental: | ST-3 | DGF_0011_01 | Stone et al. | FIGS. 1A-B | SRR3115458 |
| toxigenic | Canine | | | 2016 | and | |
| control | | | | | FIGS. 7A-B, | |
| ST-2: | Clinical: | ST-2 | HS- | This study | FIGS. 1A-B | SRR6841703 |
| human | Human | | FS_0042_01 | | and | |
| isolate | | | | | FIGS. 7A-B, | |
| ST-2: | Environmental: | ST-2 | DGF_0196_10 | Stone et al. | FIGS. 1A-B | SRR3115498 |
| canine | Canine | | | 2016 | and | |
| isolate | | | | | FIGS. 7A-B, | |
| ST-2: soil | Environmental: | ST-2 | ARDPond_soil | This study | FIGS. 7A-B, | SRR7309419 |
| isolate | Soil | | | | | |
| ST-2: | Clinical: | ST-2 | HS- | This study | FIGS. 7A-B, | SRR7309421 |
| human | Human | | FS_0177_02 | | | |
| isolate 2 | | | | | | |
| ST-1: | Clinical: | ST-1 | HS- | This study | FIGS. 7A-B, | SRR6890723 |
| human | Human | | FS_0043_01 | | | |
| isolate | | | | | | |

### Epithelial Cells

To address whether major differences exist between human and canine epithelial cells in response to C. difficile toxins (Goal #2), human colonic epithelial cells (Caco-2) and primary canine colonic epithelial cells (cat # D-6047) were purchased from American Type Culture Collection (ATCC, Manassas, Va., USA) and Cell Biologics (Cell Biologics, Inc., Chicago, Ill., USA), respectively. Caco-2 cells were maintained in EMEM (Eagle's Minimum Essential Medium) cell culture media (cat #30-2003, ATCC, Manassas, Va., USA) supplemented with 10% fetal bovine serum (FBS), 100 U/mL penicillin, 100 μg/mL streptomycin, and 2% HEPES (4-(2-hydroxyethyl)-1-piperazineethanesulfonic acid) buffer, whereas the canine cells were maintained in Complete Epithelial Cell Medium (cat # M6621, Cell Biologics, Inc., Chicago, Ill., USA) supplemented with 0.1% insulin-transferrin-selenium (ITS), 0.1% epidermal growth factor (EGF), 1% L-glutamine, 1% antibiotic-antimycotic solution, and 20% FBS.

C. difficile Toxin Supernatant Preparation and Western Blot

Individual C. difficile colonies were inoculated in 5 mL of brain heart infusion bacterial broth medium supplemented with 0.03% L-cysteine (BHIS) and incubated under anaerobic growth conditions at 37° C. for 5 days. Cultures were then centrifuged at 2000×g for 5 minutes and approximately 4 mL of bacterial supernatant (containing the toxins and other proteins) was filtered through a 0.2 micron filter to remove any incidental bacterial cells. Extracellular toxin production was validated for each strain by western blot using an antibody that binds to both C. difficile toxin A and B (abID # ab211057, ABCAM, Cambridge, Mass., USA). It is important to note that this does not enable the detection/quantification of TcdA and TcdB separately, but rather provides an estimate of the combined production of both toxins. A single, wide band corresponding to the molecular weight of these toxins (TcdB=270 kDa and TcdA=308 kDa) was observed on the membrane as expected based on presence/absence of tcdB gene for each isolate (image not shown), and absent from the ST-3: non-toxigenic control.

### Transepithelial Electrical Resistance (TEER) Assay

Tight junction integrity of Caco-2 and canine cell monolayers was assessed by measuring transepithelial electrical resistance (TEER) as previously described but with minor modifications. Approximately 1×10⁵ cells were seeded into each well (24 well ThinCert™ cell culture inserts, 0.4 μM pore diameter, 3.36 cm² culture surface; Greiner Bio-One, Monroe, N.C., USA) with 250 μL of the previously described cell culture media in the apical chamber and 800 μL in the basal-lateral chamber. Full differentiation and 100% confluency of the Caco-2 and canine cells was achieved by culturing for 12 days and 9 days, respectively at 37° C. with 5% CO2, which was confirmed by plateauing of the TEER readings. TEER measurements were captured using an Epithelial Volt-Ohm Meter Millicell ERS (EMD Millipore, Billerica, Mass., USA).

To assess the effects of these C. difficile supernatants on tight junction integrity, 50 μL of filtered bacterial supernatant was added into the apical chamber of each cell culture insert. TEER measurements were taken immediately before the addition of bacterial supernatant (hour 0), and also at hours 4, 24, and 48 post exposure. Sample reactions were run in triplicate and three measurements were taken at each time point and averaged. For each replicate, the standard error was calculated and is displayed as error bars in the figure. TEER values for each time point were expressed as an averaged proportion of electrical resistance to time

$$0 h ( y = \frac{Avg . TEER @ xhr}{Avg . TEER @ 0 hr} ) .$$

Additionally, in an effort to control for instrument variation at the different time points, all proportions were normalized to the TEER measurements of the untreated control cells (i.e. max TEER) at every time-point. To demonstrate that reductions in TEER measurements (i.e. tight junction breakdown) observed during these treatments reflected differences in toxin concentration exposure, we serially diluted (neat to 1:1,000) the C. difficile toxin containing supernatant derived from the ST1: toxigenic control in sterile EMEM media and exposed Caco-2 epithelial cell monolayers to 50 μL of each dilution in duplicate reactions. Finally, to determine changes due to treatment with different C. difficile supernatants and dilutions, Kruskal-Wallis tests were applied to the calculated TEER values at the 48 hour time point and p-values<0.05 were considered significant.

### Bacterial Microbiome Study Design and Sample Selection

Three separate microbiome comparisons were conducted based on the 16S rRNA gene: 1) CDI humans (CDI Human) vs. healthy humans (Healthy Human), 2) C. difficile positive canines (Canine C. diff+) vs. C. difficile negative canines (Canine C. diff−), and 3) canine and human datasets combined. These analyses were based on barcoded primer pair 515F/806R that amplifies a 254 bp section of the 16S rRNA gene (variable region V4) The resulting data were used to address two important questions regarding the effects of C. difficile on the canine gut microbiome: 1) do canine C. difficile carriers display different microbial gut compositions when compared to C. difficile negative canines, and 2) do C. difficile positive canines display deviations from homeostasis similar to those observed in diseased humans?

Fecal samples already collected were used as part of the previous C. difficile canine study and two publicly available 16S rRNA gene datasets from human microbiomes (NCBI Bioproject # PRJNA307992 and PRJNA386260). As a result of the previous study, a diverse collection of canine-derived C. difficile strains and their associated fecal samples were archived, which included representatives from 12 sequence type clusters from clade 1 of the global C. difficile phylogeny. This clade contains virulent (toxin producing) and avirulent (non-toxin producing) strains that are commonly isolated from humans, animals, and the environment. A subset (n=76) of the 216 available canine fecal samples from the previous study was selected, including: 37 C. difficile positive samples (toxigenic n−21, non-toxigenic n=16), which were grouped together to form the C. difficile positive canine cohort (see statistical analysis section below for justification of this grouping) and 39 C. difficile negative samples. Thirty one of the 37 C. difficile positive fecal samples were characterized as well-formed stools, whereas the remaining six were diarrheal. The C. difficile negative samples were selected based on multiple criteria: 1) the absence of C. difficile DNA 2) fecal composition (i.e. formed stool, which implies a healthy state), 3) robust 16S signal as measured by qPCR, which ensures adequate 16S PCR amplification for downstream sequencing and analysis, and 4) maximized geographic distribution, aimed at capturing the variation present within this canine population. The publically available datasets were selected because: 1) they represent CDI human (n=119 clinically confirmed human C. difficile index cases) and healthy human (n=211 healthy human controls 16S dataset) sequences prepared using the same methodology, and 2) sequencing primers used to generate these data were identical to those used for the canine samples (see PCR conditions below). These additional two datasets illustrate the gut microbial dysbiosis common in human CDI, while also enabling direct comparisons to the canine dataset generated for this study.

A majority of the canine samples used in this study (n=67) were collected opportunistically from the environment and therefore the time from initial deposit to −80° C. storage is unknown. This undefined sampling variable could lead to incorrect conclusions regarding bacterial abundances due to environmental growth conditions outside of the host. As such, a majority of analyses were conducted using a qualitative metric (i.e. Unweighted UniFrac based on presence/absence and phylogenetic distance of taxa) to identify bacterial community membership differences between cohorts and proceeded cautiously when using statistical methods based on bacterial abundances (i.e. LefSe analysis; see below). This conservative analysis approach was applied to all three 16S rRNA gene datasets. In addition, soil bacteria may be present in these opportunistically collected canine samples.

### Canine Fecal DNA Extractions

Nucleic acids were extracted from each fecal sample using PowerSoil® DNA extraction kits (MoBio, Carlsbad, Calif., USA) according to the manufacturers' specifications but with the following modifications aimed at increasing DNA yield: after the addition of solution C1, the samples were incubated in a hot water bath for 10 minutes at 70° C., vortexed for 30 minutes, and centrifuged at 10,000×g for 30 minutes. After the addition of solutions C2 and C3, the incubation times at 4° C. were increased to 1 hour and the post incubation centrifugation steps were increased to 10 minutes. All extractions were assessed for quality and bacterial quantity by 16S rRNA gene qPCR.

16S rRNA Gene Amplification and Library Preparation

The 16S rRNA gene V4 region was amplified using barcoded primer pair 515F/806R. PCRs were carried out in 25 μL volumes containing the following reagents (given in final concentrations): 5 μl diluted (1 part DNA: 9 parts water) template, 1× TaKaRa® Ex Taq PCR buffer (Mg2+ plus), 0.2 mM TaKaRa® dNTP mixture, 0.625 U TaKaRa® Ex Taq HS polymerase, 0.56 mg/μL BSA, and 0.4 μM of each primer. All samples were subjected to triplicate PCRs and pooled (which was employed to minimize the effects of random amplification bias on downstream analysis) using a SimpliAmp® thermocycler (Applied Biosystems, Foster City, Calif., USA) under the following conditions: 98° C. for 2 minutes to release the polymerase antibody, followed by 30 cycles of 98° C. for 20 seconds, 50° C. for 30 seconds, and 72° C. for 45 seconds. A final extension step of 72° C. for 10 minutes was then conducted to ensure completion of all fragments. Amplicons were visualized on a 2% agarose gel to verify the successful amplification of the expected ˜384 bp product. Combined replicate samples were quantified using the Qubit® HS dsDNA kit (Invitrogen, Carlsbad, Calif., USA) according to the manufacturer's instructions, and pooled for sequencing at 500 ng per sample. The final amplicon pool was purified using a QiaQuick® PCR purification kit (Qiagen, Valencia, Calif., USA) and quantified via qPCR using KAPA Library Quantification Primer Premix and qPCR Mastermix and DNA Standards for Illumina sequencing (KAPA Biosystems, Wilmington, Mass., USA). 8 pM of the final pool was loaded onto an Illumina MiSeq V2 cartridge (2×251 bp reads) along with a PhiX sequencing run control at a concentration of 20 pM (˜10% of total run) and a negative control extraction blank.

16S rRNA Gene Sequence Processing

Sequence processing for all three 16S rRNA gene datasets was performed in QIIME2 2017 versions 2.0-11.0. Paired-end 16S rRNA gene sequence data were demultiplexed using the paired-end EMP command in QIIME2. The sequence read quality plots generated during the demultiplexing step were visualized using the dada2 plot qualities command in QIIME2 for reads 1 and reads 2 and truncated to maximize the use of high quality bases only (>q30). Read 1 sequences for all three datasets were of a higher quality than read 2 and >q30 read lengths were variable from run to run, which is typical of the Illumina sequencing platform, and therefore the read 1 sequences were truncated to 225 bp (canine dataset), 200 bp (CDI human dataset), and 160 bp (healthy human dataset), whereas the read 2 sequences were truncated to 150 bp, 160 bp, and 120 bp, respectively. Sequence variants (SVs) were grouped in the QIIME2 environment based on 100% sequence identity using DADA2 and aligned using MAAFT version 7. The resulting SV tables were filtered by removing any SVs that were only present in a single sample, as SVs that are retained after this filtering step are more likely the result of a true biological phenomenon and not a sequencing artifact. To facilitate phylogenetic diversity metrics, such as Faith's Phylogenetic Diversity and Unweighted UniFrac, a rooted phylogenetic tree was built in the QIIME 2 environment using FastTree. Taxonomy was assigned to each unique SV using the Naïve Bayes classifier, which was trained on Greengenes 13_8 99% OTUs (available at https://data.qiime2.org/20170.2/common/gg-13-8-99-515-806-nbclassifier. qza). Finally, all canine sequences were collapsed in QIIME2 according to taxonomic assignment at the species level.

### Microbiome Statistical Analysis

A rarefaction analyses was conducted in QIIME2 to determine the sampling depth necessary to capture the diversity within each sample and to enable meaningful comparisons among all three 16S rRNA gene datasets used in this study. The rarefaction asymptote indicated a sampling depth of ˜5,000 sequences per canine sample and ˜4,000 sequences per human sample would be sufficient to accomplish these goals. A sampling depth of 5,228 randomly selected sequences per sample was employed to enable the inclusion of all samples in the canine dataset and samples with fewer than 5228 sequences were excluded from further analyses. Furthermore, this sampling depth was more conservative than the rarefaction analysis would suggest is necessary to capture the diversity within the human datasets, while still retaining a majority of samples in both (see Table 1).

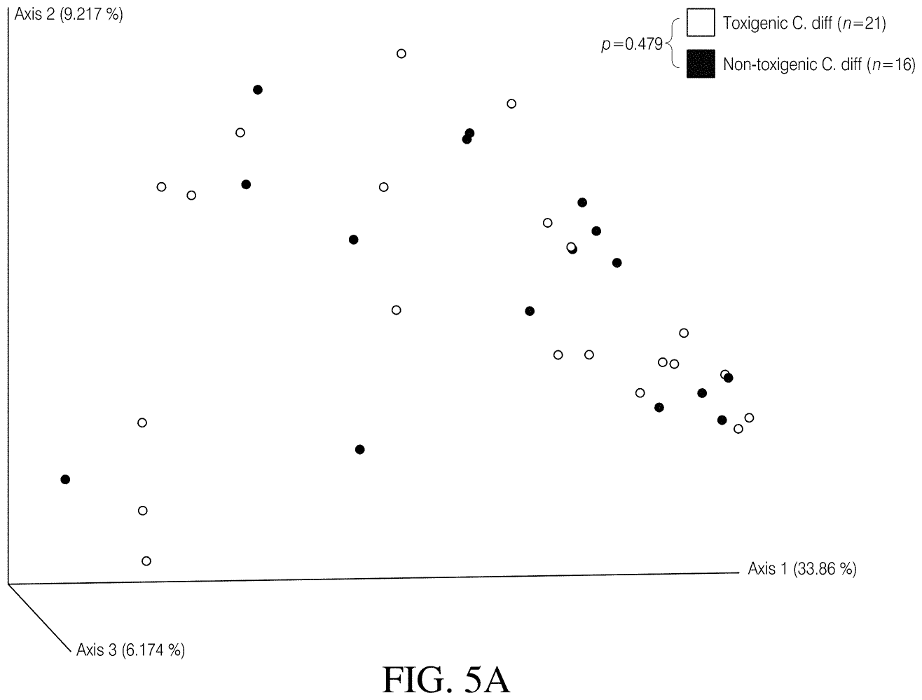

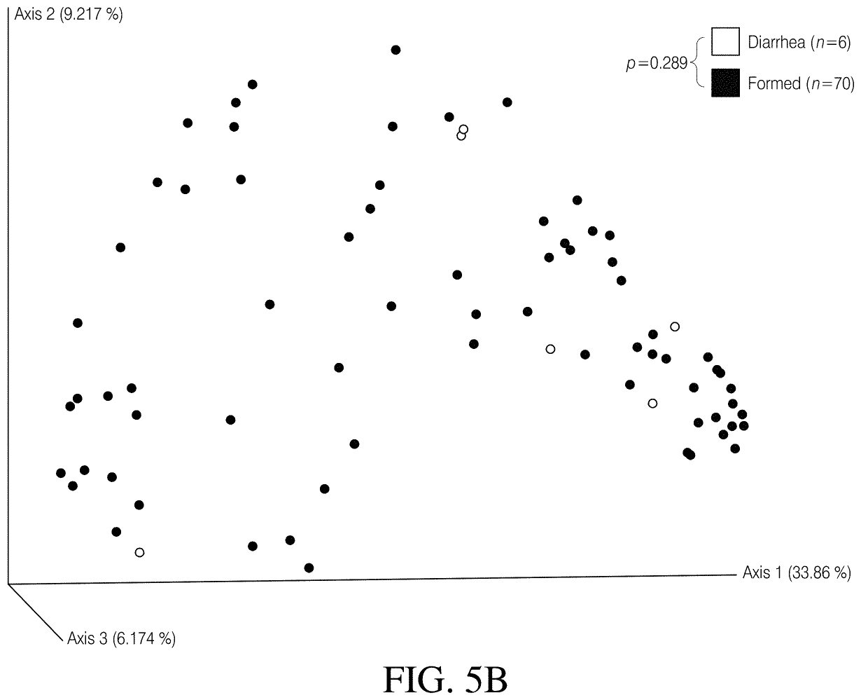

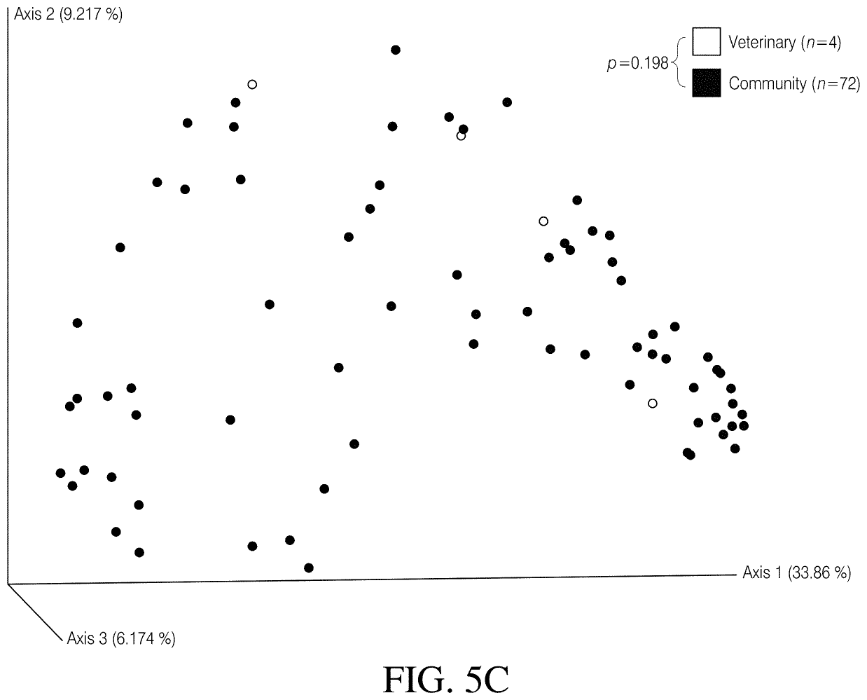

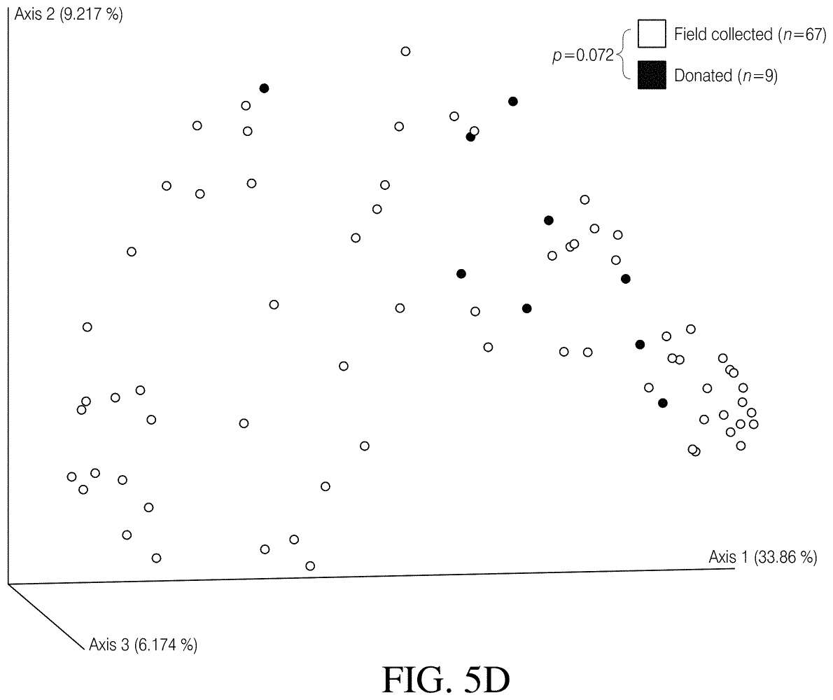

An Unweighted UniFrac dissimilarity matrix was generated in QIIME 2 and significance between cohorts was determined using permutational multivariate analysis of variance (PERMANOVA). Principal coordinates analysis (PCoA) plots were then generated using emperor. To determine changes in alpha diversity between cohorts, Kruskal-Wallis tests were conducted on calculated Faith's phylogenetic diversity and observed SVs (richness) values. Alpha diversity values were generated using the diversity coremetrics command in the QIIME 2 environment. To correct for false discovery when multiple tests were performed, the Benjamini-Hochberg method (built into the QIIME 2 coremetrics command) was employed, which reports q-values instead of p-values. Pvalues or q-values<0.05 were considered significant. It was established that grouping canine samples together into C. difficile positive (n=37) and C. difficile negative (n=39) cohorts, regardless of toxigenic status, stool composition, sampling source, and sampling strategy, was appropriate. To accomplish this we compared subgroups [toxigenic (n=21) vs. non-toxigenic (n=16); diarrhea (n=6) vs. formed stool (n=70); veterinary (n=4) vs. community collected (n=70); as well as field collected (n=67) vs. donated samples (n=9)] within and among the canine cohorts using PERMANOVA as described above and did not observe significant differences (p>0.05, PERMANOVA, FIGS. 5A-D). Furthermore, the Healthy Human cohort included 18 samples that were previously reported as having detectable levels of C. difficile reads using the Resphera Insight method. As such, the same diversity metrics described above were used to determine the clustering and diversity patterns of these C. difficile positive healthy human samples (i.e. asymptomatic carriers) and compared them to C. difficile negative samples (n=193) within the Healthy Human cohort. In Table 1 data is presented wherein three datasets were used to conduct the microbiome portion of this study. The two human datasets illustrate the effects of CDI in humans and provide a framework for making comparisons to C. difficile carriage in canines. A sampling depth of 5228 randomly selected reads per sample enabled meaningful comparisons across all three datasets, while still retaining a majority of the samples.

TABLE 1

| | | Read | | | | | |
|---|---|---|---|---|---|---|---|
| | | frequency | Total | | | | |
| | | per | features | Total | Sampling | Samples | Citation/NCBI |
| Datasets | Total reads | sample | (SVs) | samples | depth | retained | Bioproject # |
| Canines | 8,111,767 | 5228-328,954 | 749 | 76 | 5228 | 76 | Stone et al. |
| | | | | | | | 2016/PRJNA309189 |
| CDI | 1,752,349 | 1087-26,880 | 774 | 119 | 5228 | 110 | Seekatz et al. 2016/ |
| humans | | | | | | | PRJNA307992 |
| Healthy | 4,460,092 | 1753-38,201 | 1400 | 211 | 5228 | 210 | Seekatz et al. |
| humans | | | | | | | unpublished/PRJNA386260 |
| Total | 14,324,208 | 1087-328,954 | 2310 | 406 | — | 396 | |

To partition all samples used in this study (human and canine, n=396 total) into clusters (i.e. groups) based on sample similarity, the Partitioning Around Medoids (PAM) clustering algorithm was used in the R environment based on Unweighted UniFrac distances, using average silhouette width score as an indicator of the optimal number of clusters. PAM assigns membership of individual samples to a specific cluster based on the shortest distance to the theoretical center of a particular cluster and does not take into account predefined metadata categories. Silhouette scores range from −1 to 1 and higher values indicate that a sample is well assigned to a particular cluster. k values of 2, 3, and 4 were tested because these three solutions were similarly plausible, displaying silhouette scores of S(i)=0.194, 0.181, and 0.185, respectively. To provide a graphical representation of the PAM clusters and the sample assignments to these clusters, principal component analysis (PCA) and silhouette plots were generated in R.

A biomarker discovery analysis was conducted aimed at identifying biologically relevant taxa that may explain clustering patterns observed among sampling groups using linear discriminant analysis (LDA) effect size (LefSe). This analysis ranks taxa based on biological relevance and abundance (i.e. effect size), and, in essence, generates testable hypotheses regarding biologically meaningful taxa that may contribute to observed differences between cohorts. The following pairwise tests for all cohorts used in this study were performed: Canine vs. Human, CDI Human vs. Healthy Human, Canine C. diff+vs. Canine C. diff−, Canine vs. CDI Human, and Canine vs. Healthy Human. The graphical representation of LDA scores are provided as plots. These analyses were conducted through the Galaxy web-based shell (http://huttenhower.sph.harvard.edu/galaxy/) with alpha values of 0.05 for Kruskal-Wallis non-parametric sumrank test and Wilcoxon rank-sum test, which were used to identify differentially abundant taxa between groups and test for biological consistency. The threshold for the logarithmic LDA scores for discriminative features was set to 4.0. Taxa that were identified as having a significant effect size in canines (i.e. were identified as significant in every canine vs. human pairwise test regardless of C. difficile status) were subjected to further interrogation. Because other microbiome analyses described herein were based on presence/absence metrics and not bacterial abundances, the proportion of samples within each cohort that contained the three most significant taxa from this LefSe analysis were calculated and these proportions were plotted. Significance between canine groups was tested for using a Chi-Square test of independence.

### Confirming Species Identification Via Sanger Sequencing

To confirm the species identification of two biologically relevant taxonomic groups identified in this study (Goal #4), previously published primers for Clostridium hiranonis and designed new primers for Sphingobacterium faecium were used to amplify and sequence highly specific regions in both species. The “Hirano F2” and “Hirano R00” primers described by Kithara et al. were used to amplify a 403 bp fragment of the C. hiranonis 16S rRNA gene. PCRs were carried out in 10 μL volumes containing the following reagents (given in final concentrations): 2-4 ng of DNA template, 1×PCR buffer, 2.5 mM MgCl₂, 0.2 mM dNTPs, 0.8 U Platinum® Taq polymerase, and 0.4 μM of each primer. PCRs were thermocycled according to following conditions: 95° C. for 10 minutes to release the polymerase antibody, followed by 40 cycles of 94° C. for 60 seconds, 59° C. for 30 seconds, and 72° C. for 30 seconds, and a final extension step of 72° C. for 10 minutes to ensure completion of the fragments. Positive template controls and negative water controls were included on all runs. PCR products were visualized on a 2% agarose gel and amplicons of the expected size (403 bp) were treated with ExoSAP-IT (Affymetrix, Santa Clara, Calif., USA) using 1 μL of ExoSAP-IT per 7 μL of PCR product under the following conditions: 37° C. for 15 minutes, followed by 80° C. for 15 minutes. Treated products were then diluted (based on amplicon intensity) and sequenced in both directions using the same forward and reverse primers from the PCR in a BigDye® Terminator v3.1 Ready Reaction Mix. 10 μL volumes were used for sequencing reactions containing the following reagents (given in final concentrations): 5× Sequencing Buffer, 1 μL BigDye® Terminator v3.1 Ready Reaction Mix (Applied Biosystems, Foster City, Calif., USA), 1 μM primer, and 5 μL diluted PCR product. The following thermocycling conditions were used: 96° C. for 20 seconds, followed by 30 cycles of 96° C. for 10 seconds, 50° C. for 5 seconds, and 60° C. for 4 minutes.

A region within the 16S rRNA gene was identified that was conserved among three species within the genus Sphingobacterium, namely S. faecium, S. kitahiroshimense, and S. anhuiense. Primers were designed using Primer Express 3.0 (Applied Biosystems, Foster City, Calif., USA), where primer pair Sphingo16S_F (5′-TAAGTCAGTGGTGAAAGACGGC-3′), and Sphingo16S_R (5′-CGCAAACATCGAGTTATCATCG-3′) generated a 244 bp amplicon. PCRs were carried out in 10 μL volumes as described above for the C. hiranonis 16S rRNA gene and thermocycled according to following conditions: 95° C. for 10 minutes to release the polymerase antibody, followed by 38 cycles of 94° C. for 60 seconds, 65° C. for 30 seconds, and 72° C. for 30 seconds, and a final extension step of 72° C. for 10 minutes. Positive template controls and negative water controls were included on all runs. PCR products were visualized on a 2% agarose gel and amplicons of the expected size (244 bp) were treated with ExoSAP-IT and sequenced as described above, except the Sphingo16S primer set was used.

### Results

### Transepithelial Electrical Resistance (TEER) Assay and Western Blot Results

TEER: Both Human and Canine Epithelial Cells are Susceptible to C. difficile Toxins

It was first verified that human-derived Caco-2 epithelial monolayer displayed increased permeability (i.e. reduction in TEER) when exposed to three bacterial supernatants containing C. difficile toxins. Supernatant from the highly toxigenic control (ST-1: toxigenic control) led to a decrease in TEER of 75.2% by hour 4 and a complete reduction to baseline (100% decrease) by 24 hours, whereas the supernatants from canine (ST-2: canine isolate) and human (ST-2: human isolate) derived isolates had a less intense effect with reductions of 29.9% and 5.5% by hour 4, 76.7% and 44.0% by hour 24, and 83.6% and 53.3% by 48 hours, respectively (p=0.009, Kruskal-Wallis, FIG. 1A). When exposed to these same supernatants for experimental Goal #2, the canine epithelial cell monolayer displayed reduction in epithelial integrity similar to the human Caco-2 cells. The supernatant from the ST-1: toxigenic control led to a decrease in TEER of 87.3% by hour 4 and reached baseline by hour 48. The supernatants from the ST-2: canine isolate and the ST-2: human isolate caused a decrease in TEER of 78.9% and 54.4% at hour 4, and 91.1% and 89.8% at hour 24, respectively, with a reduction to baseline at hour 48 for both (p=0.016, Kruskal-Wallis, FIG. 1B). The supernatant obtained from the ST-3: nontoxigenic control led to an increase in TEER of 32.9% (canine cells) and 41.2% (Caco-2 cells) beyond the untreated epithelial monolayer at hour 48, which may indicate the presence of essential cellular nutrients and additional substrate in the bacterial supernatant.

TEER: C. difficile Toxin Production Differs Among Genetically Related Isolates Obtained from Different Hosts/Sources

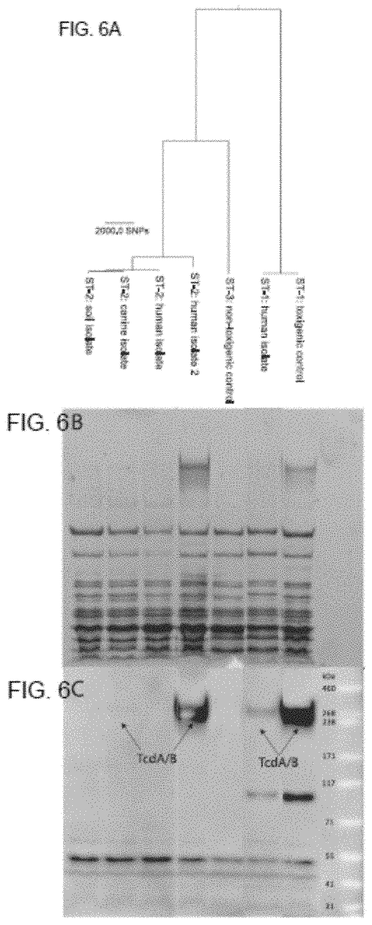

A single wide band, corresponding to the molecular weights of C. difficile toxins (TcdB=270 kDa and TcdA=308 kDa), was observed on the membrane (visible for 4 of 6 isolates) and absent from the ST-3: non-toxigenic control (FIG. 6C). Two samples (ST-2: soil isolate and ST-2: human isolate) produced toxin levels that fell below technical limits to detect with this method; however, TEER results suggested toxin production from these isolates (FIGS. 1A-B and FIGS. 7A-B). The gel images also revealed that although similar amounts of total protein were produced by all seven isolates (FIG. 6C), toxin production was highly variable among isolates (FIG. 6C). TEER experimental results reflected these toxin production differences, as measured by the effect on epithelial cell tight junction integrity, but also revealed functional similarities among related isolates (i.e. those that share a common ST) derived from different sources. Similar to the ST-2: canine isolate (TEER results reported above), the supernatant from the environmental ST-2: soil isolate caused reductions in TEER in both the Caco-2 and canine epithelial cell monolayers of 29.5% and 74.6% by hour 4, 74.8% and 90.2% by hour 24, and 82.4% and 97.0% by hour 48, respectively. In contrast, the ST-2 human isolate (ST-2: human isolate 2), which differs from the ST-2: canine isolate by 1291 SNPs, caused a decrease in TEER similar to the ST-1: toxigenic control for both cell lines (ST-1: toxigenic control; TEER results reported above), with reductions of 93.4% (Caco-2) and 95.5% (canine) by hour 4 and 99.8% and 93.1% by hour 24, respectively, and a complete reduction to baseline in both cell lines at hour 48. Interestingly, the human derived ST-1: human isolate did not display reductions that were as intense as the ST-1: toxigenic control, with moderate reductions in TEER of 19.6% (Caco-2) and 88.9% (canine) by hour 4, 82.8% and 91.4% by hour 24, and 90.1% and 100% by hour 48 (Caco-2 p=0.002, canine p=0.013, Kruskal-Wallis, FIGS. 7A-B). These TEER and western blot data from additional C. difficile isolates suggest that ST is not an accurate predictor of toxin production because an ST-2 isolate (ST-2: human isolate 2) was identified with comparable toxin production (i.e. reduction in TEER) to the ST-1: toxigenic control, and an ST-1 human isolate that could be considered “hypervirulent” based on genetic membership to ST-1 that displayed only moderate toxin production when compared to the ST-1: toxigenic control (FIG. 6A-C and FIGS. 7A-B).

### TEER: Reductions are Toxin Dose Dependent

The TEER results from the serially diluted (undiluted to 1:1000) supernatant that was derived from the ST-1: toxigenic control demonstrated that reductions in TEER were toxin dose dependent. In contrast to the exposure of Caco-2 cells to undiluted (neat) toxins, which resulted in a complete TEER reduction to baseline by hour 24, the 1:10, 1:100, and 1:1000 dilutions decreased TEER by 94.2%, 3.4%, and 0.0% by hour 24, and 96.6%, 24.2%, 0.0% by hour 48, respectively (p=0.078, Kruskal-Wallis, FIG. 8).

### Microbiome Analyses

### Microbiome: PERMANOVA and Kruskal-Wallis

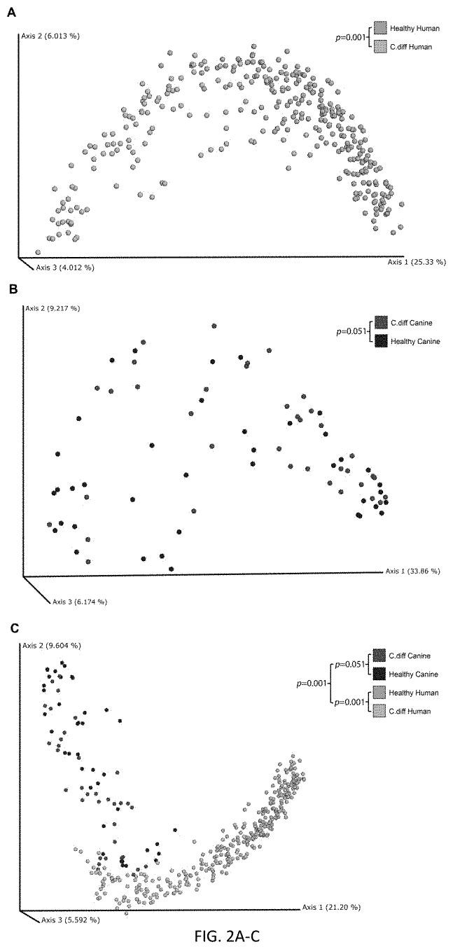

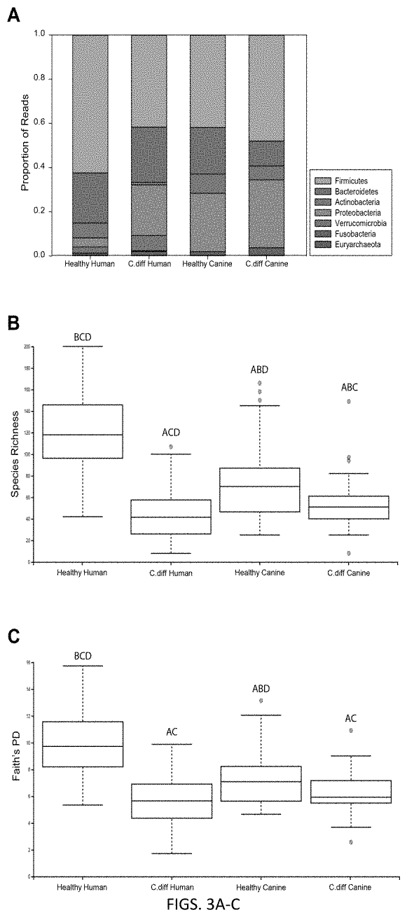

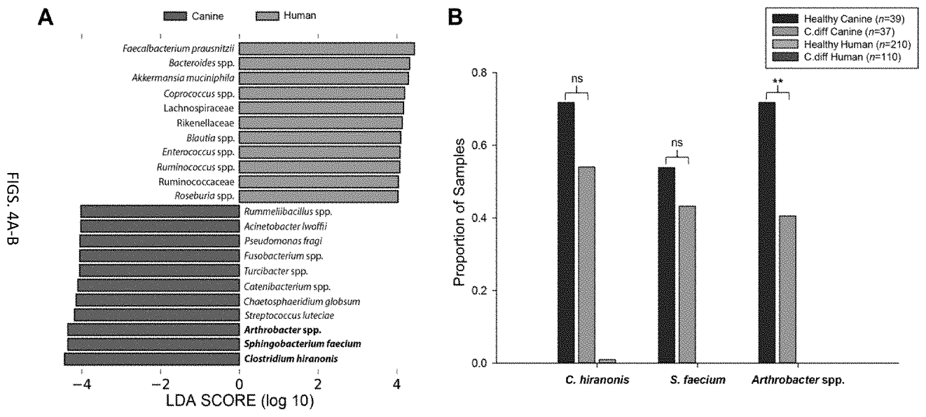

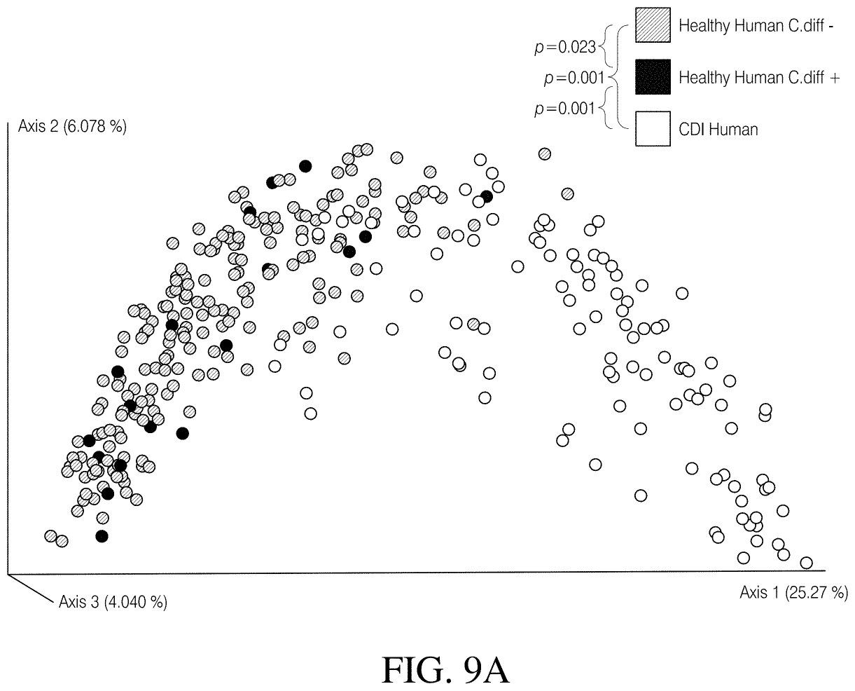

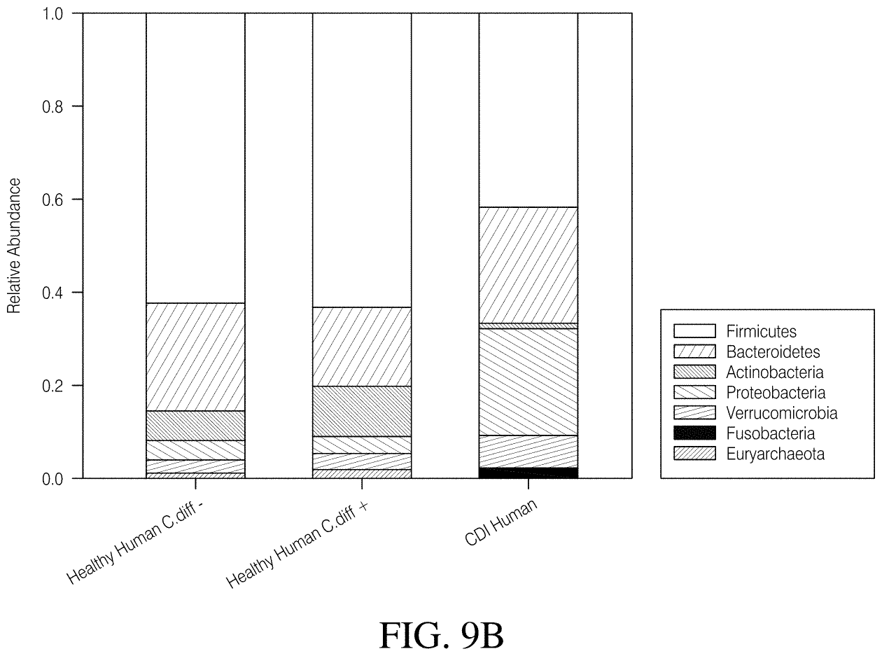

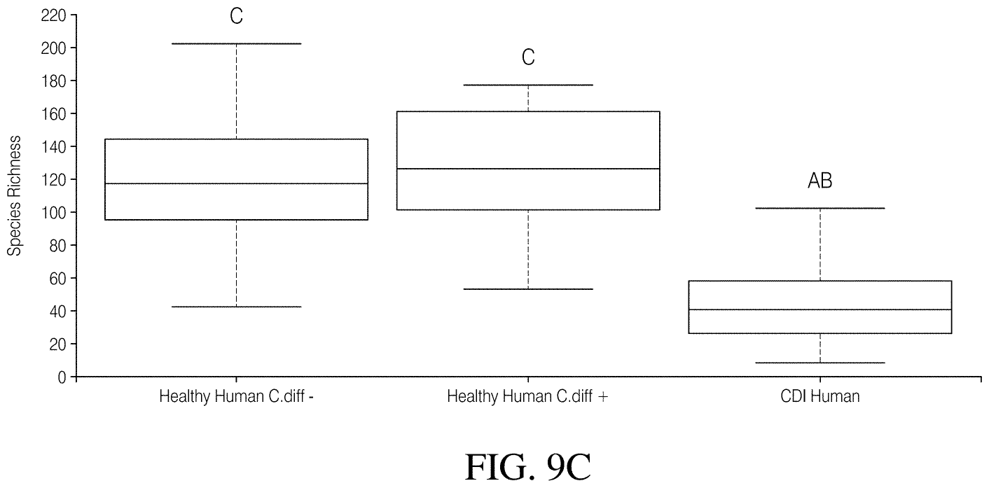

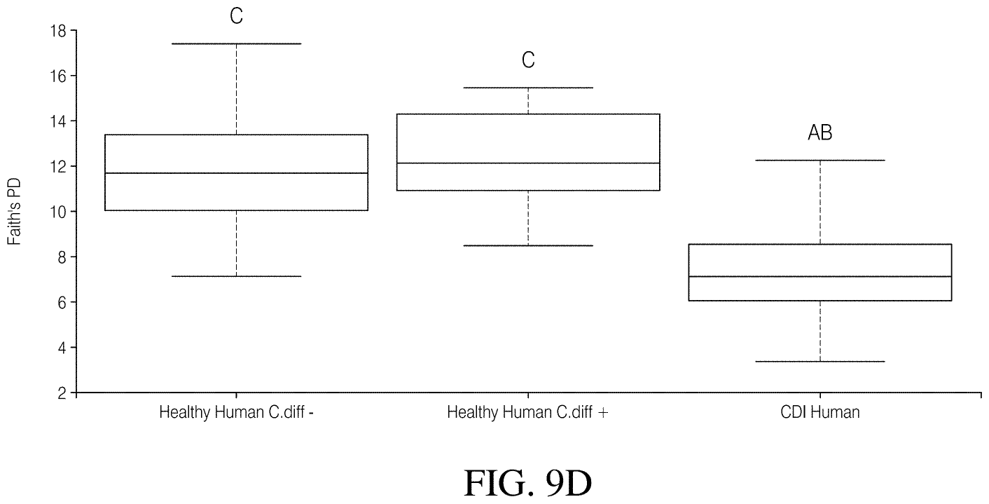

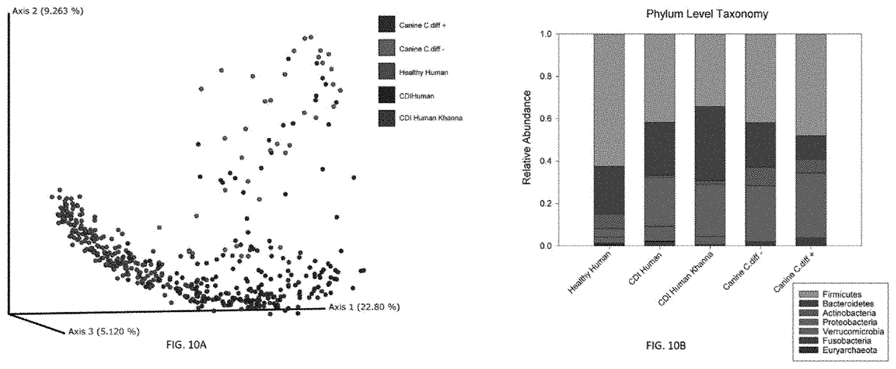

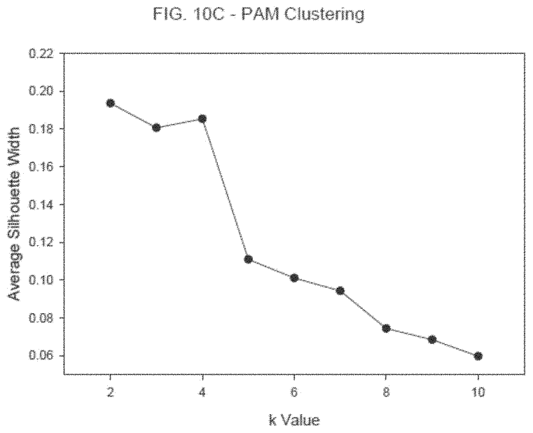

16S rRNA gene sequences were processed from 406 total samples from three datasets, which resulted in 14,324,208 high quality (>q30) sequences (range=1,087-328,954 per sample with a mean read frequency=35,281) and the identification of 2310 SVs. Samples represented by less than 5228 sequences were excluded from downstream analysis, which led to the removal of ten human samples and a final dataset of 396 samples (Table 1). The PCoA plot of Unweighted UniFrac distances illustrates a clear separation between CDI human (CDI Human) and healthy human (Healthy Human) samples (p=0.001; FIG. 2A). This pattern was marked by a percent decrease in microbial community membership in the phyla Euryarchaeota (86.6%), Actinobacter (83.3%), and Firmicutes (33.3%), but an increase in membership to Fusobacteria (99.5%), Proteobacteria (82.1%), Verrucomicrobia (59.5%), and Bacteroidetes (9.6%) in the CDI Human cohort (FIG. 3A). Furthermore, a median reduction in species richness of 76.5 (p<0.001, Kruskal-Wallis) and Faith's phylogenetic diversity of 4.11 (p<0.001, Kruskal-Wallis) was observed in the CDI Human cohort (FIGS. 3B and C). In contrast, the PCoA plot of Unweighted UniFrac distances illustrates an almost complete overlap of the Canine C. diff+(C. difficile positive) and Canine C. diff− (C. difficile negative) cohorts, and PERMANOVA did not identify community structure associated with C. difficile carriage in canines (p=0.51; FIG. 2B). An observable shift in bacterial community membership between these two cohorts at the phylum level, with increases in Fusobacteria (48.9%), Proteobacteria (13.8%), and Firmicutes (12.8%), and decreases in Verrucomicrobia (48.1%), Bacteroidetes (46.5%), Euryarchaeota (29.3%), and Actinobacteria (27.5%) may be associated with the presence of C. difficile in canines (FIG. 3A). Additionally, a median reduction in species richness (p=0.026, Kruskal-Wallis) and Faith's phylogenetic diversity (p=0.008, Kruskal-Wallis) was reported in the Canine C. diff+ cohort when compared to the Canine C. diff− cohort (FIGS. 3B and C). When the human and canine datasets were analyzed together, the PCoA plot of Unweighted UniFrac distances revealed distinct differences between all humans and all canines (p<0.001, PERMANOVA) with very little microbial overlap (FIG. 2C). Additionally, significant deviations (q<0.05) in species richness between all four datasets (i.e. CDI Human, Healthy Human, Canine C. diff+, and Canine C. diff−) was noted, as well as in Faith's phylogenetic diversity, with the exception of the CDI Human and C. diff Canine cohorts (FIGS. 3B and C). When comparing C. difficile positive healthy humans (Healthy Humans C. diff+) to C. difficile negative healthy humans (Healthy Humans C. diff−) within the Healthy Human cohort the PCoA plot and taxonomic barplots revealed that samples were interspersed and highly similar regardless of C. difficile status (FIGS. 9A and B), and furthermore, that there were no significant differences in species richness (q=0.27, Kruskal-Wallis; FIG. 9C) and Faith's phylogenetic diversity (q=0.231, Kruskal-Wallis; FIG. 9D) between these Healthy Human cohort subgroups. To ensure that the observed differences among these cohorts was the result of true biological phenomena and not the result of sample preparation and sequencing processes that may differ among lab groups and/or facilities (Table 1), an additional PCoA plot and an additional taxonomic barplot (using identical analyses as described above) were generated that included a second CDI human dataset that was processed by a third facility (n=88 clinically confirmed human C. difficile index cases, NCBI Bioproject # PRJNA342347). Read 1 and read 2 sequences were truncated to 230 bp and 140 bp, respectively, and a sampling depth of 5228 randomly selected sequences per sample was applied. The inclusion of this additional human CDI dataset (CDI Human Khanna) revealed similar clustering patterns and microbial gut compositions as the CDI Human dataset used in this study (FIGS. 10A-B), and significant deviations in species richness (q=0.179) or Faith's phylogenetic diversity (q=0.436) were not observed between these two CDI human datasets, which reinforces the observed patterns associated with host and CDI status presented above.

### Microbiome: Partitioning Around Medoids (PAM)

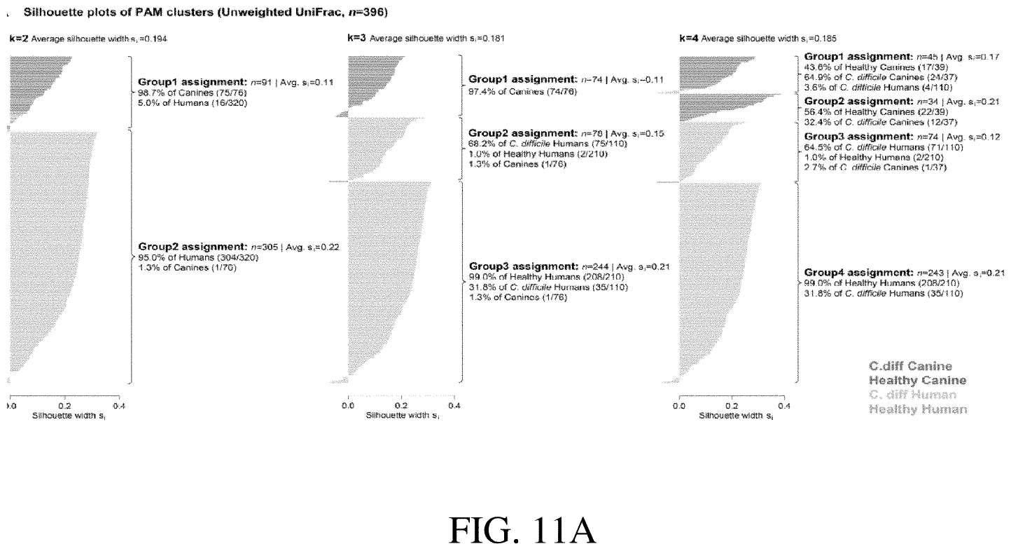

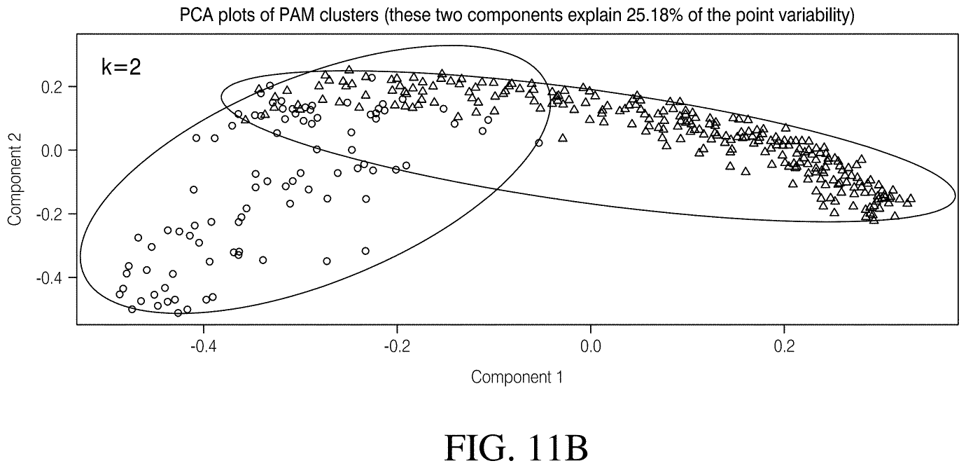

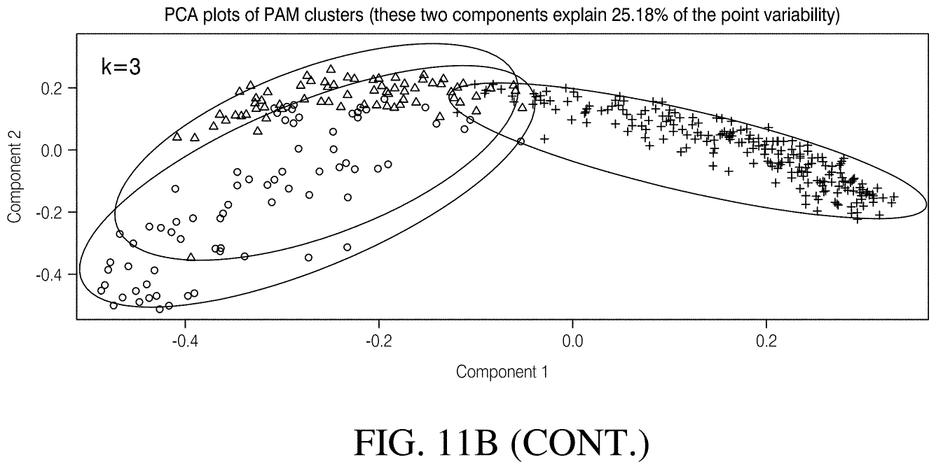

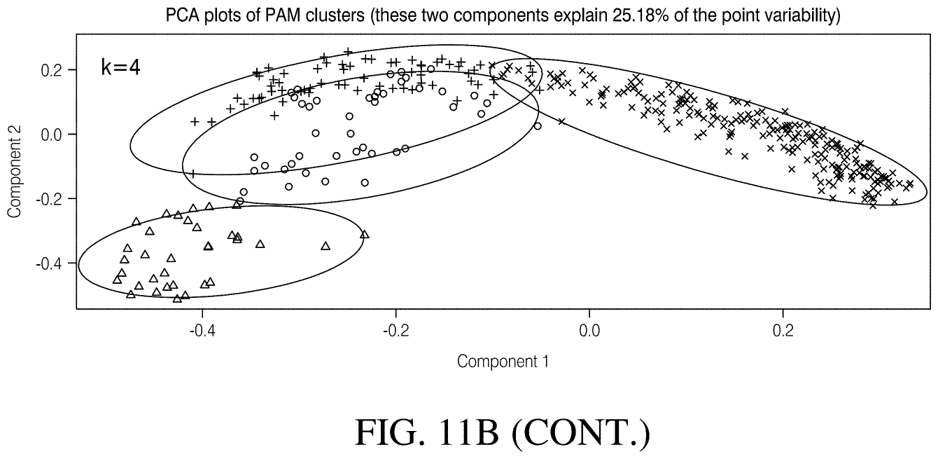

Three clustering solutions were identified as plausible based on silhouette width scores (k=2-4, mean range of 0.181-0.194; FIG. 10C) and, therefore, warranted further investigation. A clustering solution of 2 (k=2, avg. S(i)=0.194) identified structure between all canine and all human samples by assigning 98.7% (75/76) of the canine samples to a common cluster (k=2: Group 1) and 95.0% (304/320) of the human samples to a second cluster (k=2: Group 2; FIGS. 11A-B). A clustering solution of 3 (k=3, avg. S(i)=0.181) identified clusters corresponding to canines (regardless of C. difficile status), a majority of CDI humans, and healthy humans by assigning 97.4% (74/76) of the canine samples to a common cluster (k=3: Group 1), 68.2% (75/110) of the CDI Human cohort samples to a second cluster (k=3: Group 2), and 99.0% (208/210) of the Healthy Human cohort samples to a third cluster (k=3: Group 3; FIGS. 11A-B); 31.8% (35/110) of the CDI Human cohort samples were assigned to the Healthy Human cohort cluster (k=3: Group 3). Finally, a clustering solution of 4 (k=4, avg. S(i)=0.185) identified clusters corresponding to CDI humans and healthy humans by assigning 64.5% (71/110) of the CDI Human cohort samples to a common cluster (k=4: Group 3), and 99.0% (208/210) of the Healthy Human cohort samples to a separate cluster (k=4: Group 4) (FIGS. 11A-B). Additionally, 98.7% (75/76) of the canine samples assigned to one of two clusters (k=4: Group 1 and Group 2), wherein 43.6% (17/39) of the Healthy Canine cohort assigned to Group 1 and 56.4% (22/39) assigned to Group 2, and 64.9% (24/37) of the C. diff Canine cohort assigned to Group 1 and 32.4% (12/37) assigned to Group 2 (FIGS. 10A-B; Chi-Square test of independence: p=0.045). An identical percentage (31.8%) of the C. diff Human cohort samples assigned to the Healthy Human cohort cluster (k=4: Group 4) was noted as was observed at k=3, which is perhaps a reflection of the variability and unpredictability of the deviation from homeostasis that occurs in the human gut associated with CDI.

### Microbiome: Linear Discriminant Analysis (LDA) Effect Size (LefSe)

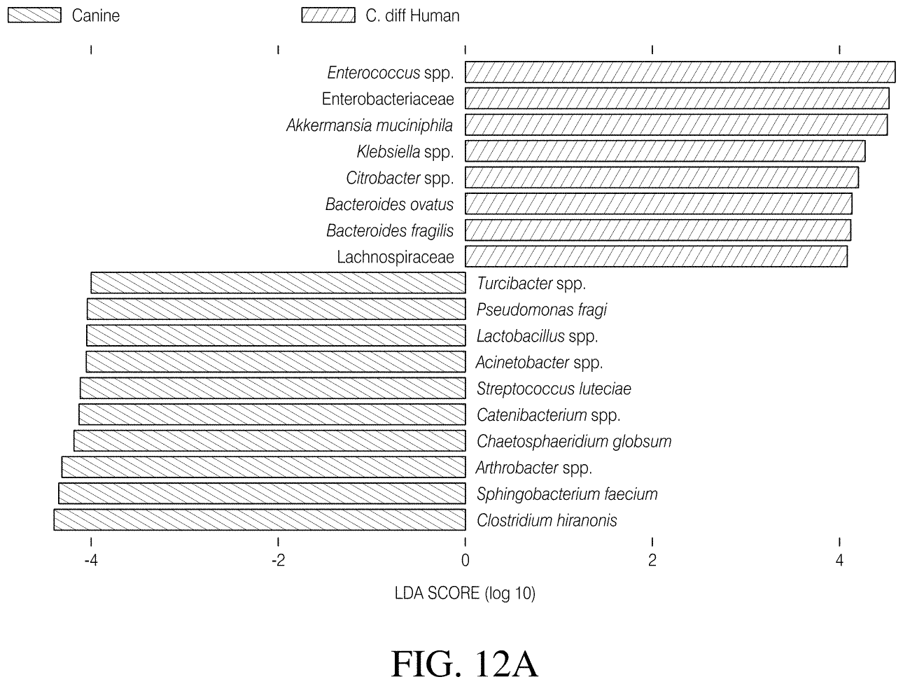

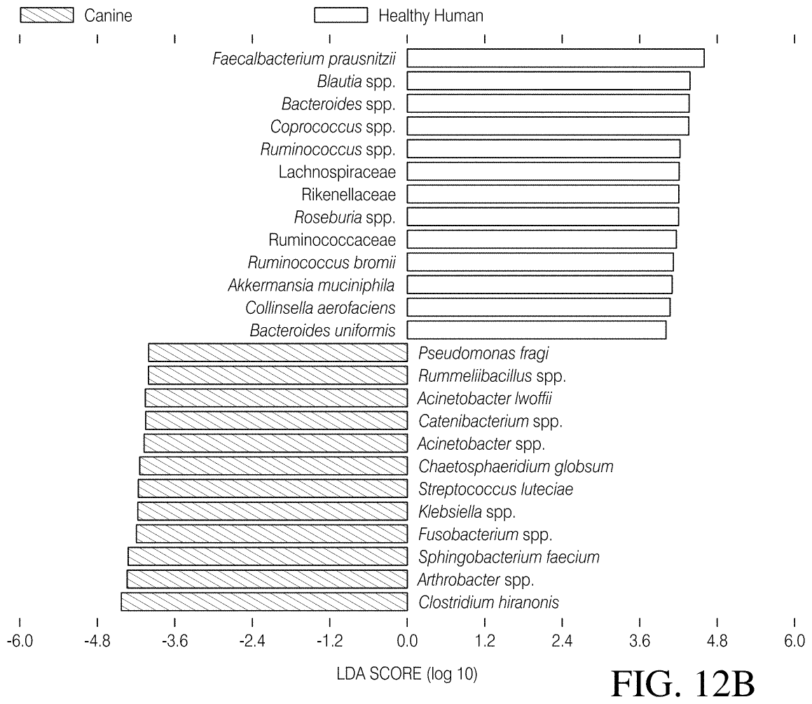

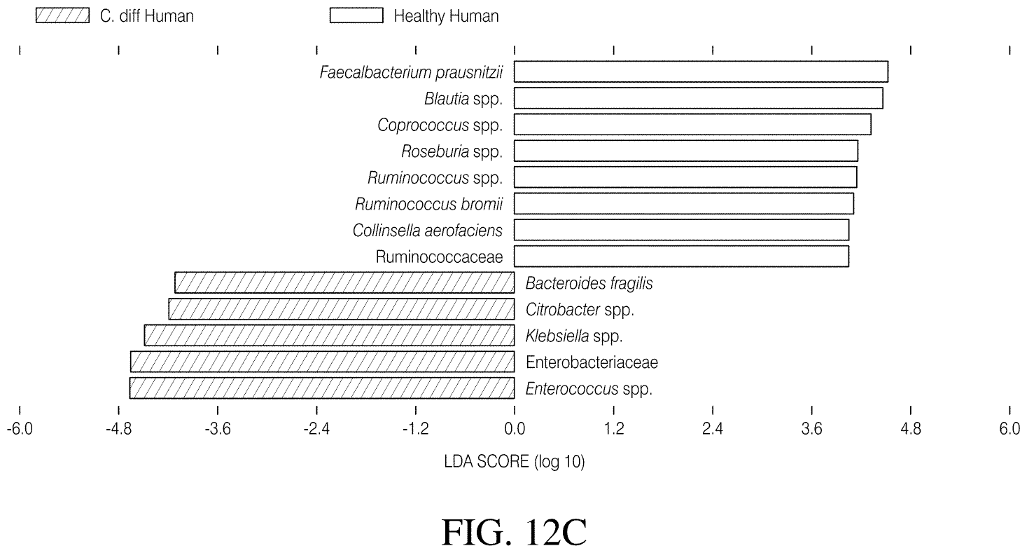

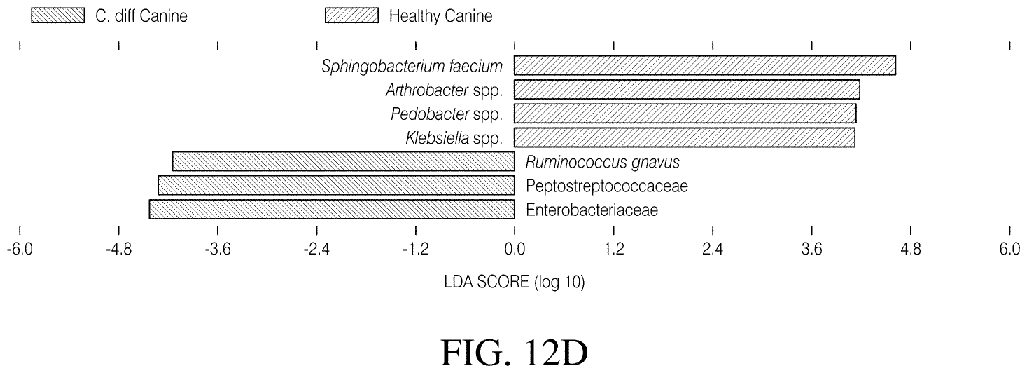

The LefSe analysis identified twenty-two bacterial taxa that differentiated canines from humans at a logarithmic LDA score of 4.0 (FIG. 4A). Eleven taxa were associated with canines, eight of which (Pseudomonas fragi, Acinetobacter spp., Streptococcus luteciae, Catenibacterium spp., Chaetosphaeridium globsum, Arthrobacter spp., Sphingobacterium faecium, and Clostridium hiranonis) were also associated with canines in two other analyses (Canine vs. CDI Human, and Canine vs. Healthy Human; FIGS. 12A and B). Eight shared taxa were focused on and it was found that three (C. hiranonis, S. faecium, and Arthrobacter spp.) were identified as having the largest effect size in all three of these analyses (FIG. 4A and FIGS. 12A and B). Additional investigation of these three taxa revealed that: 1) C. hiranonis comprised 4.93% of the total reads in the combined canine cohorts, but only 0.05% of the reads in the combined human cohorts; 2) S. faecium represented 4.64% of the total canine reads, but was completely absent from the human reads; and 3) Arthrobacter spp. accounted for 3.13% of canine reads and was also absent from the human reads (read counts provided in FIG. 4B). Additionally, C. hiranonis sequences were detected in 48 of 76 (63.16%) canines but only two of 320 (0.63%) humans, and S. faecium and Arthrobacter spp. sequences were present in 37 of 76 (48.68%) and 43 of 76 (56.60%) of canines, respectively (FIG. 4B). Because Sphingobacterium spp. are known soil microbes and, therefore, the presence of S. faecium in canine feces may be due to contamination from soil, the presence of these sequences in donated and veterinary samples (n=9) was looked for, because the period of direct contact with soil was superficial for these samples (i.e. samples were immediately collected post deposit) and the time from initial deposit to 4° C. temporary storage was known (<24 h). The S. faecium sequence was detected in 4/9 (44.4%) of these samples, which is similar to the rate of detection in the opportunistically collected samples (33/67, or 49.3%). Finally, Chi-Square tests of independence did not identify an association between either the proportion of C. hiranonis or S. faecium positive samples in the Canine C. diff+ versus the Canine C. diff− cohorts (p>0.05) but did identify an association between these two cohorts regarding the proportion of Arthrobacter spp. (p=0.01: see FIG. 4B). Of the major taxa that could explain the deviations associated with C. difficile status in canines observed during this study (i.e. PAM analysis including Canine C. diff+ vs. Canine C. diff− cohorts; FIGS. 11A-B), this analysis identified seven of potential interest. Enterobacteriaceae, Peptostreptococcaceae, and Ruminococcus gnavus were associated with the Canine C. diff+ cohort, whereas Sphingobacterium faecium, Arthrobacter spp., Pedobacter spp., and Klebsiella spp. were associated with Canine C. diff− cohort (FIG. 12D). At a logarithmic LDA score of 4.0, the LefSe analysis of the CDI Human vs. Healthy Human cohorts revealed eight taxa associated with a healthy state (Feacalbacterium prausnitzii, Blautia spp., Coprococcus spp., Roseburia spp., Ruminococcus spp., Ruminococcus bromii, Collinsella aerofaciens, and Ruminococcaceae) and five associated with CDI (Enterococcus spp., Enterobacteriaceae, Klebsiella spp., Citrobacter spp., and Bacteroides fragilis; FIG. 12C).

### Microbiome: Confirming Species Identification

Using specific primers C. hiranonis from 46 of 48 (95.8%) canine samples that contained putative C. hiranonis reads and Sphingobacterium spp. (S. faecium, S. kitahiroshimense, or S. anhuiense) from 30 of 37 (81.1%) was successfully amplified and sequenced from samples that contained putative S. faecium reads. In both instances the 16S rRNA gene fragments were a perfect sequence match to the type strains for these species (C. hiranonis strain TO-931 Genbank accession # AB023970.1 and S. faecium strain DSM-11690 Genbank accession # NR_025537.1). The two and seven samples, respectively, that failed the species confirmation PCR and sequencing had notably low levels of C. hiranonis (53 and 218 sequencing reads present) and S. faecium (5e31 sequencing reads present) and, therefore, likely fell below the technical limit to amplify with these PCRs.

### Discussion

Results of further exploration of the possibility of canines as asymptomatic carriers of C. difficile, identify specific bacterial taxa that may contribute to the infection resistance phenotype in canines, and identify potential canine colonization and infection resistance mechanisms (i.e. bacterial functions or species) that may overlap with humans are described herein. Together, the results obtained enable one to draw broad conclusions regarding asymptomatic C. difficile carriage in canines, examine the potential of canine strains to cause human CDI, and generate new hypotheses about the mechanisms that may be responsible for colonization and infection resistance in canines and relate those to hypothesized mechanisms of CDI resistance in humans. It has been demonstrated that C. difficile isolated from a canine is capable of producing toxins and that, when exposed to identical toxins, canine colonic cells are no less susceptible to disruptions of epithelial tight junctions than human cells. Clostridioides difficile toxins cause disease by perforation of the colonic epithelium, and finding of tight junction breakdown with both canine-derived and human-derived isolates suggests that there are no fundamental differences in the ability of canine-derived C. difficile to produce toxins in vitro (FIGS. 1A and B).

Furthermore, analysis of total protein and toxin production from seven diverse C. difficile isolates suggest that variability in toxin production is more closely associated with genetic content of the isolate, rather than source of the isolate (FIGS. 6A-C). However, the possibility that the current or previous environment and/or metabolic substrates could play some role in toxin production cannot be excluded. Experimental results revealed that, in vitro, canine epithelial monolayers are susceptible to tight junction breakdown as a result of exposure to C. difficile toxins (FIGS. 1A and B), which suggests that there should be similar disease outcomes in canines as in other mammals if exposed to comparable toxin concentrations. However, the majority of canine samples used in this study did not reflect a diseased state (as indicated by stool consistency, potentially suggesting mechanisms of CDI resistance in canines. An important limitation of this study is that it is unknown whether these C. difficile positive canines were colonized or simply transient carriers at the time of sample collection. Transient carriage of C. difficile would likely have limited effects (if any) on the bacterial composition of the gut due to the lack of toxin production in vivo and, thus, could also provide an explanation for the “healthy” appearance of some of these C. difficile positive canine samples. The severe microbial dysbiosis associated with human CDI (FIG. 2A) was not reflected in the canine gut microbiome (FIG. 2B).

Independent analyses both captured the commonly reported deviations to the human microbiome associated with CDI and the previously described differences between human and canine gut microbiomes (FIG. 2C and FIGS. 11A-B). However, in contrast to the human cohorts, patterns between the canine cohorts were far more ambiguous, portraying, at best, weak clustering associated with C. difficile status (FIGS. 11A-B). Furthermore, when the human and canine datasets were visualized together (PCoA and silhouette plots), the lack of grouping associated with C. difficile carriage in canines was even more compelling (FIGS. 2B and C and FIGS. 11A-B) because it illustrated a tremendous amount of diversity among canine samples but also illustrated that this diversity was not strongly correlated with the presence of C. difficile. Interestingly, two additional studies identified similar patterns of highly diverse microbiomes in canines, and one of these looked for correlations between specific bacterial taxa and multiple GI disorders but did not identify an association between the detection of toxigenic C. difficile and acute diarrhea, although the sample size was very low. That said, subtle changes (i.e. measurable, but intermediate decreases in species richness and diversity) were identified to the canine gut microbiome (FIGS. 3A-C), which may reflect a compositional shift associated with asymptomatic carriage of C. difficile in canines. Indeed, asymptomatic C. difficile carriage in humans has been shown to decrease the gut bacterial diversity in a similar fashion. Interestingly, microbiome analyses revealed that canine guts (regardless of C. difficile status) may be more similar to humans with CDI than to healthy humans (FIGS. 2C and 3A-C). The overall similarity among canine gut microbiomes with and without C. difficile (FIG. 2B), along with the resemblance to patterns of asymptomatic carriage in human guts (FIGS. 9A-D), suggests that these canines were not impacted by CDI at the time of sample collection. This finding is in line with an assessment of asymptomatic carriage in these canines based upon the non-diarrheal composition of a majority of the C. difficile positive stool samples (31 of 37), because dysbiosis has been shown to be an indicator of gastrointestinal disease in canines. Furthermore, it corroborates the suspicions of asymptomatic C. difficile carriage in canines suggested by multiple other studies and compiled in a 2011 review, which also described a small experiment where Koch's postulates for C. difficile as a canine pathogen could not be fulfilled. These findings may suggest that the canine gut contains microbial components that protect against C. difficile proliferation and subsequent toxin production and/or that there may be canine-specific immune responses that mitigate the effects of C. difficile toxins on the colon epithelium. A member of the canine gut microbiome, C. hiranonis, was identified that synthesizes secondary bile acids; a metabolite that has been shown to provide resistance to C. difficile in mice (FIGS. 4A-B and FIGS. 12A and B). Clostridium hiranonis is a member of Clostridium cluster XI and has the unique characteristic of bile acid 7a-dehydoxylating activity; converting primary bile acids cholic acid (CA) and chenodeoxycholic acid (CDCA) into secondary bile acids deoxycholic acid (DCA) and lithocholic acid (LCA), respectively. Six members of the genus Clostridium have been characterized as having this function (C. hiranonis, C. scindens, C. bifermentans, C hylemonae, C. leptum, and C. sordellii), of which C. hiranonis and C. scindens have been described as having elevated levels of 7a-dehydoxylating activity (at least ten times higher than the other four Clostridium spp.). The secondary bile acid DCA has been experimentally shown in vitro to inhibit growth of C. difficile, and C. scindens (a DCA producer and functional analogue to C. hiranonis) inhibited C. difficile in mice via bile acid mediated resistance. In humans, secondary bile acid converting bacteria comprise only ˜0.0001% of total colonic microbiota. Here, C. hiranonis was identified in two samples from the Healthy Human cohort, but absent from the CDI Human cohort (FIG. 4B); it has been previously identified as a component of the healthy human gut microbiome. These patterns suggest the potential use of C. hiranonis as a CDI therapeutic aimed at restoring the function of the healthy human gut but more studies will be needed. Although we did not quantify the relative abundance of C. hiranonis sequences in our canine samples (due to caveats associated with storage conditions, see methods above), we did observe them in over 60% of our combined canine samples (FIG. 4B), which suggests it is a normal component of the canine gut microbiome. In fact, C. hiranonis specifically, as well as other members of Clostridium cluster XI, have previously been described as common and significant contributors to the microbiome of canines.

Also identified was a bacterial species of the genus Sphingobacterium that is known to be associated with regulating homeostasis between microbes and host, which also may aid in CDI resistance in canines (FIGS. 4A-B and FIGS. 12A and B).

The production of antibacterial peptides and the initiation of innate immune responses have both been proposed as important mechanisms for combating C. difficile colonization and the effects of its toxins on the host epithelium. A high frequency of combined canine samples (>48%) that contained S. faecium sequences (FIG. 4B), a member of the genus Sphingobacterium known for having high concentrations of sphingophospholipids as cellular lipid components, was identified. Sphingolipids (of which sphingophospholipids are a class) have been suggested to play an important role in regulating balance between host and microbes. It has also been suggested that sphingolipids display antibacterial properties via the formation of sphingolipid-enriched rafts (i.e. small mammalian cellular membrane domains, enriched with sphingolipids and cholesterol), which have been shown to lead to phagocytosis of Pseudomonas aeruginosa and, therefore, may aid in the host's defense against infection. Species belonging to the genus Sphingobacterium have been mostly isolated from clinical specimens, soil, and other environmental sources, but only S. faecium has been isolated from the feces of mammals (Bos sprunigenius taurus and Hippopotamus amphibious). Therefore, even though independent species confirmation sequencing did not provide the resolution needed to distinguish between three species within the genus Sphingobacterium (S. faecium, S. kitahiroshimense, and S. anhuiense), data suggests S. faecium as the most likely species present in these samples. Due to the role that sphingolipids may play in host defense and immune response activation, it is possible that the likely presence of S. faecium in these samples is associated with CDI resistance, which warrants the exploration into the functional role that this bacterium may play in canines.

### CONCLUSIONS

When taken together, previous studies reporting the identification of genetically indistinguishable C. difficile from humans and canines, paired with the experimental and descriptive results presented here, suggest that canines may be asymptomatic reservoirs for this devastating human pathogen. It has been shown that, in vitro, a canine derived C. difficile isolate produces toxins in a similar fashion to a genetically similar human isolate. Additionally, it has been shown that colonic epithelial cells from a canine are susceptible to tight junction breakdown as a result of exposure to C. difficile toxins, which excludes the possibility that canine epithelial cells are incompatible with C. difficile toxins but, instead, suggests that features of the canine gut microbiome and/or initiation of a species specific immune response could be responsible for colonization and infection resistance in canines. Indeed, microbiome results provide supporting evidence for asymptomatic carriage of potentially disease-causing strains of C. difficile in these canine samples because deviations from homeostasis that are indicative of CDI were not observed. Furthermore, two species of bacteria were identified that could be associated with infection resistance in canines (Clostridium hiranonis and Sphingobacterium faecium); one of which supports the hypothesis of bile acid mediated resistance to C. difficile in humans.

Finally, findings suggest that the specific mechanisms of CDI resistance employed in the canine gut, once definitively identified, could provide insights into improved treatment options for human CDI.

Literature references relating to C. difficile include the following, the contents of which are hereby incorporated by reference in their entireties and are hereby made a part of this specification: J. S. Martin, T. M. Monaghan, M. H. Wilcox, Clostridium difficile infection: epidemiology, diagnosis and understanding transmission, Nat. Rev. Gastroenterol. Hepatol. 13 4 (2016) 206-216; K. E. Dingle, D. Griffiths, X. Didelot, J. Evans, A. Vaughan, M. Kachrimanidou, et al., Clinical Clostridium difficile: clonality and pathogenicity locus diversity, PLoS One 6 5 (2011), e19993; M. Rupnik, M. H. Wilcox, D. N. Gerding, Clostridium difficile infection: new developments in epidemiology and pathogenesis, Nat. Rev. Microbiol. 7 7 (2009) 526-536; D. W. Eyre, M. L. Cule, D. J. Wilson, D. Griffiths, A. Vaughan, L. O'Connor, et al., Diverse sources of C. difficile infection identified on whole-genome sequencing, N. Engl. J. Med. 369 13 (2013) 1195-1205; A. Gupta, S. Khanna, Community-acquired Clostridium difficile infection: an increasing public health threat, Infect. Drug Resist. 7 (2014) 63-72; L. H. Gould, B. Limbago, Clostridium difficile in food and domestic animals: a new foodborne pathogen? Clin. Infect. Dis.: an official publication of the Infectious Diseases Society of America 51 5 (2010) 577-582; C. W. Knetsch, T. R. Connor, A. Mutreja, S. M. van Dorp, I. M. Sanders, H. P. Browne, et al., Whole genome sequencing reveals potential spread of Clostridium difficile between humans and farm animals in The Netherlands, 2002 to 2011, Euro Surveill.: bulletin Europeen sur les maladies transmissibles=European communicable disease bulletin 19 45 (2014) 20954; N. E. Stone, L. C. Sidak-Loftis, J. W. Sahl, A. J. Vazquez, K. B. Wiggins, J. D. Gillece, et al., More than 50% of Clostridium difficile isolates from pet dogs in Flagstaff, USA, carry toxigenic genotypes, PLoS One 11 10 (2016), e0164504; U.S. Pet, Ownership & Demographics Sourcebook, 2012; S. L. Lefebvre, L. G. Arroyo, J. S. Weese, Epidemic Clostridium difficile strain in hospital visitation dog, Emerg. Infect. Dis. 12 6 (2006) 1036-1037; J. Clooten, S. Kruth, L. Arroyo, J. S. Weese, Prevalence and risk factors for Clostridium difficile colonization in dogs and cats hospitalized in an intensive care unit, Vet. Microbiol. 129 (1-2) (2008) 209e214; C. Vincent, A. R. Manges, Antimicrobial use, human gut microbiota and Clostridium difficile colonization and infection, Antibiotics (Berlin) 4 3 (2015) 230-253; S. Schmitz, J. Suchodolski, Understanding the canine intestinal microbiota and its modification by pro-, pre- and synbiotics—what is the evidence? Vet. Med. Sci. 2 2 (2016) 71-94; J. S. Suchodolski, M. E. Markel, J. F. Garcia-Mazcorro, S. Unterer, R. M. Heilmann, S. E. Dowd, et al., The fecal microbiome in dogs with acute diarrhea and idiopathic inflammatory bowel disease, PLoS One 7 12 (2012), e51907; Y. Vazquez-Baeza, E. R. Hyde, J. S. Suchodolski, R. Knight, Dog and human inflammatory bowel disease rely on overlapping yet distinct dysbiosis networks, Nat Microbiol 1 (2016) 16177; A. M. Seekatz, J. Aas, C. E. Gessert, T. A. Rubin, D. M. Saman, J. S. Bakken, et al., Recovery of the gut microbiome following fecal microbiota transplantation, mBio 5 3 (2014); C. Ghose, Clostridium difficile infection in the twenty-first century, Emerg. Microb. Infect. 2 (2013), e62; E. C. Keessen, W. Gaastra, L. J. Lipman, Clostridium difficile infection in humans and animals, differences and similarities, Vet. Microbiol. 153 (3e4) (2011) 205-217; M. M. Riggs, A. K. Sethi, T. F. Zabarsky, E. C. Eckstein, R. L. P. Jump, C. J. Donskey, Asymptomatic carriers are a potential source for transmission of epidemic and nonepidemic Clostridium difficile strains among long-term care facility residents, Clin. Infect. Dis. 45 8 (2007) 992-998; E. van Nood, A. Vrieze, M. Nieuwdorp, S. Fuentes, E. G. Zoetendal, W. M. de Vos, et al., Duodenal infusion of donor feces for recurrent Clostridium difficile, N. Engl. J. Med. 368 5 (2013) 407-415; Z. Kassam, C. H. Lee, Y. Yuan, R. H. Hunt, Fecal microbiota transplantation for Clostridium difficile infection: systematic review and meta-analysis, Am. J. Gastroenterol. 108 4 (2013) 500-508; C. G. Buffie, V. Bucci, R. R. Stein, P. T. McKenney, L. Ling, A. Gobourne, et al., Precision microbiome reconstitution restores bile acid mediated resistance to Clostridium difficile, Nature 517 7533 (2015) 205-208; A. Ofosu, Clostridium difficile infection: a review of current and emerging therapies, Ann. Gastroenterol. 29 2 (2016) 147-154; M. C. Abt, P. T. McKenney, E. G. Pamer, Clostridium difficile colitis: pathogenesis and host defence, Nat. Rev. Microbiol. 14 10 (2016) 609-620; K. M. Ng, J. A. Ferreyra, S. K. Higginbottom, J. B. Lynch, P. C. Kashyap, S. Gopinath, et al., Microbiota-liberated host sugars facilitate post-antibiotic expansion of enteric pathogens, Nature 502 7469 (2013) 96-99; I. Jarchum, M. Liu, C. Shi, M. Equinda, E. G. Pamer, Critical role for MyD88-mediated neutrophil recruitment during Clostridium difficile colitis, Infect. Immun. 80 9 (2012) 2989-2996; I. Jarchum, M. Liu, L. Lipuma, E. G. Pamer, Toll-like receptor 5 stimulation protects mice from acute Clostridium difficile colitis, Infect. Immun. 79 4 (2011) 1498-1503; M. C. Rea, C. S. Sit, E. Clayton, P. M. O'Connor, R. M. Whittal, J. Zheng, et al. C. D. Thuricin, A posttranslationally modified bacteriocin with a narrow spectrum of activity against Clostridium difficile, Proc. Natl. Acad. Sci. U.S.A. 107 20 (2010) 9352-9357; P. A. Sutton, S. Li, J. Webb, K. Solomon, J. Brazier, Y. R. Mahida, Essential role of toxin A in C. difficile 027 and reference strain supernatant-mediated disruption of Caco-2 intestinal epithelial barrier function, Clin. Exp. Immunol. 153 3 (2008) 439-447; K. Matter, M. S. Balda, Functional analysis of tight junctions, Methods 30 3 (2003) 228-234; J. W. Sahl, D. Lemmer, J. Travis, J. M. Schupp, J. D. Gillece, M. Aziz, et al., NASP: an accurate, rapid method for the identification of SNPs in WGS datasets that supports flexible input and output formats, Microb. Genom. 2 8 (2016), e000074; J. G. Caporaso, C. L. Lauber, W. A. Walters, D. Berg-Lyons, C. A. Lozupone, P. J. Turnbaugh, et al., Global patterns of 16S rRNA diversity at a depth of millions of sequences per sample, Proc. Natl. Acad. Sci. U.S.A. 108 (Suppl 1) (2011) 4516-4522; J. G. Caporaso, C. L. Lauber, W. A. Walters, D. Berg-Lyons, J. Huntley, N. Fierer, et al., Ultra-high-throughput microbial community analysis on the Illumina HiSeq and MiSeq platforms, ISME J. 6 8 (2012) 1621-1624; D. Griffiths, W. Fawley, M. Kachrimanidou, R. Bowden, D. W. Crook, R. Fung, et al., Multilocus sequence typing of Clostridium difficile, J. Clin. Microbiol. 48 3 (2010) 770-778; A. M. Seekatz, K. Rao, K. Santhosh, V. B. Young, Dynamics of the fecal microbiome in patients with recurrent and nonrecurrent Clostridium difficile infection, Genome Med. 8 1 (2016) 47; N. Daquigan, A. M. Seekatz, K. L. Greathouse, V. B. Young, J. R. White, High-resolution profiling of the gut microbiome reveals the extent of Clostridium difficile burden, NPJ Biofilms Microbiomes 3 (2017) 35; E. K. Cope, A. N. Goldberg, S. D. Pletcher, S. V. Lynch, Compositionally and functionally distinct sinus microbiota in chronic rhinosinusitis patients have immunological and clinically divergent consequences, Microbiome 5 (1) (2017) 53; J. G. Caporaso, J. Kuczynski, J. Stombaugh, K. Bittinger, F. D. Bushman, E. K. Costello, et al., QIIME allows analysis of high-throughput community sequencing data, Nat. Methods 7 5 (2010) 335-336; B. J. Callahan, P. J. McMurdie, M. J. Rosen, A. W. Han, A. J. Johnson, S. P. Holmes, DADA2: high-resolution sample inference from Illumina amplicon data, Nat. Methods 13 7 (2016) 581-583; K. Katoh, D. M. Standley, MAFFT multiple sequence alignment software version 7: improvements in performance and usability, Mol. Biol. Evol. 30 4 (2013) 772-780; M. N. Price, P. S. Dehal, A. P. Arkin, FastTree 2-approximately maximumlikelihood trees for large alignments, PLoS One 5 3 (2010), e9490; C. Lozupone, R. Knight, UniFrac: a new phylogenetic method for comparing microbial communities, Appl. Environ. Microbiol. 71 12 (2005) 8228-8235; Y. Vazquez-Baeza, M. Pirrung, A. Gonzalez, Knight R. EMPeror, A tool for visualizing high-throughput microbial community data, GigaScience 2 (1) (2013) 16; M. Arumugam, J. Raes, E. Pelletier, D. Le Paslier, T. Yamada, D. R. Mende, et al., Enterotypes of the human gut microbiome, Nature 473 7346 (2011) 174-180; N. Segata, J. Izard, L. Waldron, D. Gevers, L. Miropolsky, W. S. Garrett, et al., Metagenomic biomarker discovery and explanation, Genome Biol. 12 6 (2011) R60; M. Kitahara, M. Sakamoto, Y. Benno, PCR detection method of Clostridium scindens and C. hiranonis in human fecal samples, Microbiol. Immunol. 45 3 (2001) 263-266; S. Khanna, E. Montassier, B. Schmidt, R. Patel, D. Knights, D. S. Pardi, et al., Gut microbiome predictors of treatment response and recurrence in primary Clostridium difficile infection, Aliment Pharmacol. Ther. 44 7 (2016) 715-727; J. Y. Chang, D. A. Antonopoulos, A. Kalra, A. Tonelli, W. T. Khalife, T. M. Schmidt, et al., Decreased diversity of the fecal Microbiome in recurrent Clostridium difficile-associated diarrhea, J. Infect. Dis. 197 3 (2008) 435-438; H. Matsuyama, H. Katoh, T. Ohkushi, A. Satoh, K. Kawahara, I. Yumoto, Sphingobacterium kitahiroshimense sp. nov., isolated from soil, Int. J. Syst. Evol. Microbiol. 58 (Pt 7) (2008) 1576-1579; W. Wei, Y. Zhou, X. Wang, X. Huang, R. Lai, Sphingobacterium anhuiense sp. nov., isolated from forest soil, Int. J. Syst. Evol. Microbiol. 58 (Pt 9) (2008) 2098-2101; M. Kitahara, F. Takamine, T. Imamura, Y. Benno, Clostridium hiranonis sp. nov., a human intestinal bacterium with bile acid 7alpha-dehydroxylating activity, Int. J. Syst. Evol. Microbiol. 51 (Pt 1) (2001) 39-44; H. Oyaizu, K. Komagata, Chemotaxonomic and phenotypic characterization of the strains of species in the flavobacterium-cytophaga complex, J. Gen. Appl. Microbiol. 27 1 (1981) 57-107; D. E. Voth, J. D. Ballard, Clostridium difficile toxins: mechanism of action and role in disease, Clin. Microbiol. Rev. 18 2 (2005) 247-263; M. Rupnik, Is Clostridium difficile-associated infection a potentially zoonotic and foodborne disease? Clinical microbiology and infection: the official publication of the European Society of Clinical Microbiology and Infectious Diseases 13 5 (2007) 457-459; S. J. Song, C. Lauber, E. K. Costello, C. A. Lozupone, G. Humphrey, D. Berg-Lyons, et al., Cohabiting family members share microbiota with one another and with their dogs, Elife 2 (2013), e00458; S. Handl, S. E. Dowd, J. F. Garcia-Mazcorro, J. M. Steiner, J. S. Suchodolski, Massive parallel 16S rRNA gene pyrosequencing reveals highly diverse fecal bacterial and fungal communities in healthy dogs and cats, FEMS Microbiol. Ecol. 76 2 (2011) 301-310; M. C. Rea, O. O'Sullivan, F. Shanahan, P. W. O'Toole, C. Stanton, R. P. Ross, et al., Clostridium difficile carriage in elderly subjects and associated changes in the intestinal microbiota, J. Clin. Microbiol. 50 3 (2012) 867-875; L. H. Zhang, D. F. Dong, C. Jiang, Z. Li, X. F. Wang, Y. B. Peng, Insight into alteration of gut microbiota in Clostridium difficile infection and asymptomatic C. difficile colonization, Anaerobe 34 (2015) 1-7; L. G. Arroyo, S. A. Kruth, B. M. Willey, H. R. Staempfli, D. E. Low, J. S. Weese, PCR ribotyping of Clostridium difficile isolates originating from human and animal sources, J. Med. Microbiol. 54 2 (2005) 163-166; J. A. Winston, C. M. Theriot, Impact of microbial derived secondary bile acids on colonization resistance against Clostridium difficile in the gastrointestinal tract, Anaerobe 41 (2016) 44-50; C. M. Theriot, V. B. Young, Interactions between the gastrointestinal microbiome and Clostridium difficile, Annu. Rev. Microbiol. 69 (2015) 445-461; Q. Li, C. L. Lauber, G. Czarnecki-Maulden, Y. Pan, S. S. Hannah, Effects of the dietary protein and carbohydrate ratio on gut microbiomes in dogs of different body conditions, mBio 8 (2017) 1; L. J. Heung, C. Luberto, M. Del Poeta, Role of sphingolipids in microbial pathogenesis, Infect. Immun. 74 1 (2006) 28-39; E. Yabuuchi, T. Kaneko, I. Yano, C. W. Moss, N. Miyoshi, Sphingobacterium gennov, Sphingobacterium-spiritivorum comb nov, Sphingobacterium-multivorum comb nov, Sphingobacterium-mizutae sp-nov, and flavobacterium-indologenes sp-nov-glucose-nonfermenting gram-negative rods in cdc group-iik-2 and group-iib, Int. J. Syst. Bacteriol. 33 3 (1983) 580-598; H. Grassme, V. Jendrossek, A. Riehle, G. von Kurthy, J. Berger, H. Schwarz, et al., Host defense against Pseudomonas aeruginosa requires ceramide-rich membrane rafts, Nat. Med. 9 3 (2003) 322-330; Y. S. Fu, F. Hussain, N. Habib, I. U. Khan, X. Chu, Y. Q. Duan, et al., Sphingobacterium soli sp nov., isolated from soil, Int. J. Syst. Evol. Microbiol. 67 7 (2017) 2284-2288.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. A composition selected from the group consisting of a probiotic composition, a probiotic product combination, a pharmaceutical composition, a pharmaceutical product combination, a dietary supplement, a food, and combinations thereof, the composition comprising at least one bacterium selected from the genera consisting of Clostridium and/or Sphingobacterium, formulated for oral or rectal administration to a mammalian subject.

2. The composition of claim 1, comprising at least one bacterium selected from the genera Clostridium and at least one bacterium selected from the genera Sphingobacterium.

3. The composition of claim 1, comprising Clostridium hiranonis.

4. The composition of claim 1, comprising Sphingobacterium faecium.

5. The composition of claim 1, comprising Clostridium hiranonis and Sphingobacterium faecium.

6. The composition of claim 1, comprising at least two bacteria selected from the genera consisting of Clostridium and/or Sphingobacterium, wherein each of the bacteria is provided in a different composition.

7. The composition of claim 1, comprising 10⁴ cfu or more of the at least one bacterium.

8. The composition of claim 1, comprising 10⁶ cfu or more of the at least one bacterium.

9. The composition of claim 1, wherein the composition is in a form suitable for oral administration.

10. The composition of claim 1, wherein the composition is in a form for use as a probiotic for oral administration as a dietary supplement.

11. The composition of claim 1, wherein the subject is human.

12. The composition of claim 1, wherein the subject is canine.

13. A method of treating, preventing, or ameliorating a Clostridium difficile infection, comprising:
administering the composition of claim 1 to a subject in need thereof, whereby a Clostridium difficile infection is treated, prevented, or ameliorated.

14. The method of claim 13, wherein the Clostridium difficile infection is treated.

15. The method of claim 13, wherein the subject is infected with Clostridium difficile.

16. The method of claim 13, wherein the subject is not infected with Clostridium difficile but is exposed to Clostridium difficile before administering the composition or after administering the composition.

17. The method of claim 13, further comprising, prior to the administering, selecting the subject as being within a class of subjects that are in need of receiving the composition.

18. The method of claim 17, wherein the selecting comprises identifying the subject as having a Clostridium difficile infection.

19. The method of claim 13, further comprising administering an antibiotic to the subject prior to administering the composition, whereby the antibiotic reduces a total quantity of gut bacteria of the subject by at least 80% prior to the administering the composition.
